(19) United States
(12) Patent Application Publication
(10) Pub. No.: US 2005/0037430 A1
(43) Pub. Date: Feb. 17, 2005

(54) Methods and uses for protein breakdown products

(75) Inventors: Nisar Ahmed Khan, Rotterdam (NL); Robbert Benner, Barendrecht (NL)

Correspondence Address: TRASK BRITT, P.O. BOX 2550, SALT LAKE CITY, UT, 84110, US

(73) Assignee: Biotempt B.V., Koekange (NL)

(21) Appl. No.: 10/821,240

(22) Filed: Apr. 8, 2004

Related U.S. Application Data
(63) Continuation-in-part of application No. 10/753,510, filed on Jan. 7, 2004.
Application No. 10/753,510 is a continuation-in-part of application No. 10/028,075, filed on Dec. 21, 2001.
Application No. 10/753,510 is a continuation-in-part of application No. PCT/NL02/00639, filed on Apr. 10, 2003.
Application No. 10/753,510 is a continuation of application No. 10/262,522, filed on Sep. 30, 2002.
Application No. 10/262,522 is a continuation of application No. PCT/NL01/00259, filed on Mar. 3, 2001.

(30) Foreign Application Priority Data
Mar. 29, 2000 (EP) 00201139.3
Oct. 4, 2001 (EP) 01203748.7

(51) Int. Cl. G01N 33/53; G06F 19/00; G01N 33/48; G01N 33/50

(52) U.S. Cl. 435/7.1; 702/19

(57) ABSTRACT

Where it was generally thought that the smallest breakdown products of proteins had no specific biological function on their own, it now emerges that the body may utilize the normal process of proteolytic breakdown to generate important compounds such as gene-regulatory compounds. For instance, certain short breakdown products of hCG (i.e., short peptides which can easily be synthesized, if needed modified, and used as a pharmaceutical composition) exert a major regulatory activity on pro- or anti-inflammatory cytokine cascades that are governed by a family of crucial transcription factors, the NF-κB family, which generally regulate the expression of genes involved in the body's immune response.

### CROSS-REFERENCE TO RELATED APPLICATION

This application is a continuation in part of co-pending application, U.S. Ser. No. 10/753,510, filed Jan. 7, 2004, the contents of the entirety of which is incorporated by this reference.

### TECHNICAL FIELD

The invention relates generally to biotechnology, and more specifically to methods of generating and screening small molecules, such as di-mer, tri-mer, 4-mer, 5-mer, 6-mers, 7-mers, and a database generated by screening the small molecules and methods of using the database.

### BACKGROUND

U.S. Pat. No. 5,380,668 to Herron (Jan. 10, 1995), the contents of the entirety of which are incorporated by this reference, discloses, among other things, various compounds having the antigenic binding activity of human chorionic gonadotropin (hCG). The oligopeptides disclosed therein are disclosed generally for use in diagnostice methods.

Various patents and patent applications to Gallo et al. (e.g., U.S. Pat. No. 5,677,275 (corresponding to WO 96/04008 A1), U.S. Pat. No. 5,877,148 (also corresponding to WO 96/04008 A1), WO 97/49721 A1, U.S. Pat. No. 6,319,504 (corresponding to WO 97/49373), U.S. Patent Application 2003/0049273 A1 (also corresponding to WO 97/49373), U.S. Pat. No. 5,968,513 (corresponding to WO 97/49418), U.S. Pat. No. 5,997,871 (corresponding to WO 97/49432), U.S. Pat. No. 6,620,416, U.S. Pat. No. 6,596,688, WO 01/11048 A2, WO 01/10907 A2., and U.S. Pat. No. 6,583,109) relate to various oligopeptides and their use in, among other things, “inhibiting HIV infection”, “treating or preventing HIV infection”, “treating or preventing cancer”, “treating or preventing a condition characterized by loss of body cell mass”, “treating or preventing a condition associated with pathological angiogenesis”, “treating or preventing hematopoietic deficiency”, “ex vivo gene therapy”, “expanding blood cells in vitro”, and/or “providing blood cells to a subject”.

The current invention relates to the body's innate way of modulating important physiological processes and builds on insights reported in PCT International Publications WO99/59617 and WO01/72831 and PCT International Application PCT/NLO2/00639, the contents of the entirety of all of which are incorporated herein by this reference. These applications describe small gene-regulatory peptides that are present in pregnant women and are derived from proteolytic breakdown of placental gonadotropins, such as hCG. These breakdown products are often only about 4 to 6 amino acids long and were shown to have unsurpassed immunological activity that is exerted by regulating expression of genes encoding inflammatory mediators such as cytokines. Surprisingly, it was found that breakdown of hCG provides a cascade of peptides that helps maintain a pregnant woman's immunological homeostasis. These peptides balance the immune system to assure that the mother stays immunologically sound while her fetus does not get prematurely rejected during pregnancy, but instead is safely carried until its time of birth.

During pregnancy, the maternal system suppresses maternal rejection responses directed against the fetus. Paradoxically, during pregnancy, often the mother's resistance to infection is increased and she is found to be better protected against the clinical symptoms of various autoimmune diseases such as rheumatism and multiple sclerosis. The protection of the fetus thus cannot be interpreted as only a result of immune suppression. Each of the above three applications has provided insights by which the immunological balance between protection of the mother and protection of the fetus can be understood.

### DISCLOSURE OF THE INVENTION

Where it was generally thought that the smallest breakdown products of proteins have no specific biological function on their own, it now emerges that the body may utilize the normal process of proteolytic breakdown to generate important compounds such as gene-regulatory compounds. In particular, certain short breakdown products of hCG (i.e., short peptides which can easily be synthesized, if needed modified, and used as a pharmaceutical composition) exert a major regulatory activity on pro- or anti-inflammatory cytokine cascades that are governed by a family of crucial transcription factors, the NF-κB family, which generally regulate the expression of genes involved in the body's immune response.

In an exemplary embodiment, the invention provides a method for identifying one or more peptides; and/or determining the activity of one or more peptides, comprising, e.g., screening a peptide to determine the activity of the peptide; analyzing the results; and/or identifying one or more proteins having immunoregulatory activity and/or gene regulatory activity. Optionally, the results may be stored in a database, wherein the database may be sorted by one or more desired characteristic, for example, production of nitric oxide (NO). In addition, the database may allow searching (e.g., by means of a computer), form a remote location, such as a second computer, wherein at least part of the search result may be transmitted to the searcher via a network.

In another embodiment, the invention provides compiling and/or organizing data and/or properties for one or more peptides in a database. In another embodiment, software that performs correlative database searching is used.

The invention also provides a method of accessing information from a collection of data, including receiving a query and generating results to the query. Generating a result to the query may include storing hierarchical information generated from the collection of data, and applying search rules to the collection of data to produce a canonical non-terminal representation of the data.

In an exemplary embodiment, generating results includes applying search rules to the query to produce a query canonical form and matching the query canonical form to a canonical non-terminal representation of the data.

In yet another embodiment, the invention provides a method for conducting a drug discovery business, comprising: i) determining the identity of a compound that modulates development of the systemic inflammatory response, release of other inflammatory mediators (e.g., IL-1-α, IL-1-β, IL-6, TNF-α, LIF, IFN-γ, OSM, CNTF, TGF-β, GM-CSF, IL-11, IL-12, IL-17, IL-18, IL-8 and a variety of other chemokines), regulation of members of the nuclear factor-κB (NF-κB) family, accentuation or protection from sepsis, nitrate production, nitric oxide (NO) production, and/or glucose tolerance; ii) conducting therapeutic profiling of the compound identified in step i), or further analogs thereof, for efficacy and toxicity in animals; and, iii) formulating a pharmaceutical preparation including one or more compounds identified in step ii) as having an acceptable therapeutic profile. Such business method can be further extended by including an additional step of establishing a distribution system for distributing the pharmaceutical preparation for sale, and may optionally include establishing a sales group for marketing the pharmaceutical preparation. Optionally, determining the identity of a compound may comprise searching a database of the invention.

The instant invention also provides a business method comprising: i) by suitable methods described herein, determining the identity of a compound that modulates a systemic inflammatory response, release of other inflammatory mediators (e.g., IL-1-α, IL-1-β, IL-6, TNF-α, LIF, IFN-γ, OSM, CNTF, TGF-β, GM-CSF, IL-11, IL-12, IL-17, IL-18, IL-8 and a variety of other chemokines), regulation of members of the nuclear factor-KB (NF-κB) family, sepsis, nitrate production, nitric oxide (NO) production, and/or glucose tolerance ii) licensing, to a third party, the rights for further drug development of one or more compounds identified in step i).

The instant invention also provides a business method comprising: i) by suitable methods determining the identity of the polypeptide and the nature of the treatment or effect associated with the polypeptide, the methods optionally include searching a database of the invention; ii) licensing, to a third party, the rights for further drug development of the polypeptide.

In another embodiment, the invention provides a method of screening a peptide and derivatives thereof, for activity. Optionally, the result of a screen is entered into a database. In another embodiment, the peptide and/or derivative are arranged on a chip or array or in a multiwell format. In yet another embodiment, a peptide array or multiwell format is screened using methods known in the art and/or disclosed herein to determine one or more activities of a peptide.

### BEST MODE FOR CARRYING OUT THE INVENTION

As described in PCT International Publication No. WO 03/029292 A2 (published Apr. 10, 2003), PCT International Publication No. WO 01/72831 A2 (published Oct. 4, 2001), and U.S. Patent Application Publications 20020064501 Al (published May 30, 2002), 20030119720 A1 (published Jun. 26, 2003), 20030113733 A1 (published Jun. 19, 2003), and 20030166556 A1 (published Sep. 4, 2003), the contents of all of which are incorporated by this reference, compositions containing purified or isolated oligopeptides described therein have immunoregulatory activity useful in, for example, the treatment of sepsis and other disease states and conditions. They also have gene regulatory activities.

The invention includes a method of screening compounds, including a purified or isolated peptide consisting of particular four to eight amino acid segments of the sequence MTRVLQGVLPALPQVVC (SEQ ID NO:44); and derivatives thereof. Immunoregulatory activity may be determined by measuring a peptides's ability to modulate production of NO by a cell. In one embodiment, the compositions have the ability to decrease shock in a subject (e.g., a mammal) undergoing sepsis.

In one embodiment, the amino acid segment includes a tetrameric sequence (e.g., corresponding to the LQVG (SEQ ID NO:1)) portion of SEQ ID NO:44, i.e.,
Xaa₁ Xaa₂ Xaa₃ Xaa₄
wherein Xaa₁ is a substituted or unsubstituted non-polar amino acid selected from the group consisting of Ala and Leu; Xaa₂ is a substituted or unsubstituted amino acid selected from the group consisting of Gln, Pro, and Ala; Xaa₃ is a substituted or unsubstituted Gly; and Xaa₄ is a substituted or unsubstituted non-polar amino acid selected from the group consisting of Val and Ala. For instance, the peptide could be selected from the group consisting of LQGV (SEQ ID NO:1), the derivative AQGV (SEQ ID NO:2), the derivative LQGA (SEQ ID NO:19), the derivative LAGV (SEQ ID NO:26), and the derivative LPGC (SEQ ID NO:41).

In another embodiment, the segment is six amino acids long, and comprises the sequence
Xaa₁ Xaa₂ Pro Ala Xaa₂ Xaa₃
wherein Xaa₁ is substituted or unsubstituted Val or Ala, wherein Xaa₂ is independently selected from substituted or unsubstituted Leu or Ala, and wherein Xaa₃ is a substituted or unsubstituted Pro or Ala.

In such an embodiment, the peptide can have a formula selected from the group consisting of VLPALP (SEQ ID NO:3), the derivative ALPALP (SEQ ID NO:21), the derivative VAPALP (SEQ ID NO:22), the derivative ALPALPQ (SEQ ID NO:23), the derivative VLPAAPQ (SEQ ID NO:24), the derivative VLPALAQ (SEQ ID NO:25), the derivative VLPALA (SEQ ID NO:28), VLPALPQ (SEQ ID NO:29), the derivative VLPALPA (SEQ ID NO:31), the derivative GVLPALP (SEQ ID NO:32), and the derivative VLAALP (SEQ ID NO:117).

In another embodiment, the composition has no more than eight amino acids, and includes an amino acid sequence consisting of: Xaa₁ Xaa₂ Xaa₃ Xaa4

wherein Xaa₁ is a substituted or unsubstituted amino acid selected from the group of amino acids consisting of Ala, Leu, and Met, wherein Xaa₂ is a substituted or unsubstituted amino acid selected from the group of amino acids consisting of Gln, Thr, Ala, and Pro, wherein Xaa₃ is substituted or unsubstituted Gly or Arg, and wherein Xaa₄ is a substituted or unsubstituted amino acid selected from the group of amino acids consisting of Cys, Ala, and Val. Immunoregulatory activity may be determined by, for example, measuring the capability to modulate production of NO by a cell or affect sepsis.

In such an embodiment, the sequence may be selected from the group consisting of Leu Gln Gly Val (SEQ ID NO:1), Ala Gln Gly Val (SEQ ID NO:2), Leu Gln Gly Ala (SEQ ID NO:19), Leu Ala Gly Val (SEQ ID NO:26), Leu Pro Gly Cys (SEQ ID NO:41), or Met Thr Arg Val (SEQ ID NO:42), or a derivative thereof.

In another embodiment, the segment may be the tetramer MTRV (SEQ ID NO:42) or QVVC (SEQ ID NO:43) or a derivative thereof.

In one embodiment, a genomic or functional database includes the level of a pro inflammatory cytokine, such as pro inflammatory cytokines that are responsible for early responses, including IL-1-α, IL-1-β, IL-6, and TNF-α. Other pro inflammatory mediators include LIF, IFN-γ, OSM, CNTF, GM-CSF, IL-11, IL-12, IL-17, IL-18, IL-8 and a variety of other chemokines that chemoattract inflammatory cells, and various neuromodulatory factors. It is preferred that the results include the level of a pro inflammatory cytokine that is selected from the group of TNF-α, IFN-γ, IL-1-β and IL-6. Upon determination of pro inflammatory cytokine levels of a compound that may down regulate translocation and/or activity of pro inflammatory cytokine gene expression may be identified. In another embodiment, the relative levels of one or more transcription factor, for example, an NF-κB/Rel protein, are determined, for example, using LPS stimulated RAW264.7 cells using a multiwell format. The pro inflammatory cytokine levels, or other attributes, of the compounds may be compiled in a database and used according to the invention.

Inflammatory cytokines include IL-4, IL-10, and IL-13, IL-16, IFN-α, IL-Ira, G-CSF, and soluble receptors for TNF or IL-6. Information regarding peptides having NF-κB up regulating activity may be assayed using a peptide array or multiwell format and the results compiled in a database. For example, the activity of a peptide may be assayed in a multiwell format for modulation of NF-κB activity or NO production using RAW264.7 cells.

An essentially inflammatory condition is preferably characterized by elevated levels of at least one, but preferably at least two or three pro inflammatory cytokines, for example, produced by circulating polymorph bone marrow cells (PBMCs), such as elevated plasma or serum levels of one or more of the pro inflammatory cytokines that are responsible for early responses such as IL 1 α, IL 1 β, IL 6, and TNF α. Examples of other pro inflammatory mediators include LIF, IFN γ, OSM, CNTF, GM CSF, IL 11, IL 12, IL 17, IL 18, IL 8. As each diagnostician may have his or her own preference for testing an inflammatory mediator, it is preferred that the database be based on the level of a pro inflammatory cytokine is selected from TNF α, IFN γ, IL 1 β and/or IL 6.

Useful tests include, but are not limited to, flow cytometry assays of serum/plasma/supernatant available from BD Biosciences as a Cytometric Bead Array (CBA), for example, a CBA human Th1/Th2 cytokine kit for the measurement of IL 2, IL 4, IL 5, IL 10, TNF α and IFN γ in a single sample or available from Biosource International, the Biosource Multiplex antibody Bead Kit for measurement of IL 1β, IL 2, IL 4, IL 5, IL 6, IL 8, IL 10, IFN γ, TNF α and GM CSF in single sample or, for example, the Biosource cytoset ELISA for measurement of individual cytokines and soluble DR4.

It is also provided herein to determine HLA-DR expression, wherein decreased levels of HLA-DR antigen expression may indicate a counter inflammatory condition.

The tests and/or assays described here in may be used to screen a large number of peptides, for example, derivatives of LQGV (SEQ ID NO:1) may be synthesized and placed in a protein array. Derivatives of LQGV (SEQ ID NO:1) can include peptides produced by Ala-scanning and/or peptides produced by replacement net analysis. Derivatives may also include modified or non-natural amino acids. An array may be prepared with such peptides and used to screen the peptides, which include derivatives, for activity. Such a screen may identify peptides with one or more desired activity and/or produce positional information, for example, identification that position four (e.g., the V of SEQ ID NO:1) is preferably a non-polar amino acid, by alignment of the screen results and/or peptide sequences screened.

Organization, searching and construction of a database may be performed by any of the means known in the art (see, for example, U.S. Pat. Nos. 5,966,712; 6,278,794; 6,379,970; 6,539,102; 6,507,788; and 6,711,563, each of which is hereby incorporated by reference).

The invention further includes a pharmaceutical composition comprising a purified or isolated peptide, or acid addition salt thereof, the purified or isolated peptide identified according to the invention.

The invention provides a method for the identification of a peptide useful in the treatment of bone disease such as osteoporosis comprising administering to a subject believed to be in need of such treatment a compositon comprising a peptide, derivative or functional analogue thereof, the particular molecule capable of modulating production of NO and/or TNF-alpha by a cell.

Such compounds are particularly useful in post-menopausal women that no longer experience the benefits of being provided with a natural source of hCG and its breakdown products. Such a treatment can be achieved by systemic administration of a composition of the invention according to the invention, but local administration in joints, bursae or tendon sheaths is provided as well. The molecule can be selected from Table 6 or identified by a method desribed herein, including searching a database of the invention. The treatment comprises administering to the subject a pharmaceutical composition comprising a peptide or derivative thereof. For example, a peptide capable of reducing production of NO by a cell. In another embodiment, the composition comprises at least two peptides or derivatives thereof. For example, each peptide being capable of reducing production of NO and/or TNF-alpha by a cell. By way of example, the at least two oligopeptides may be selected from the group LQGV (SEQ ID NO:1), AQGV (SEQ ID NO:2), and VLPALP (SEQ ID NO:3) or peptides identified by a method described herein.

The methods of the invention have been used to assay several peptides according to the invention by ex vivo, in vivo, and animal assays. A beneficial effect of these oligopeptides on LPS-induced sepsis in mice, namely the inhibition of the effect of the sepsis, was observed. Immunomodulatory effects with these oligopeptides have been observed in vitro and ex vivo such as in T-cell assays showing the inhibition of pathological Th1 immune responses, suppression of inflammatory cytokines (MIF), increased production of anti-inflammatory cytokines (IL-10, TGF-beta) and immunomodulatory effects on antigen-presenting cells (APC) like dendritic cells, monocytes and macrophages.

Knowing the gene modulatory effect of the peptides of the invention, allows for the rational design of molecular mixtures that better alleviate the symptoms seen with sepsis. For example, a 1:1:1 mixture of LQGV (SEQ ID NO:1), AQGV (SEQ ID NO:2) and VLPALP (SEQ ID NO:3) was administered to primates in a gram-negative induced rhesus monkey sepsis model to test for the prevention of septic shock and found to be effective in this primate model. Accordingly, the invention provides information regarding peptides which may be used to prepare a pharmaceutical composition for the treatment of sepsis in a primate. In addition, a method for the treatment of sepsis in a primate comprising subjecting the primate to a composition of the invention, preferably selected according to the invention, preferably utilizing a mixture of such compositions. Administration of such a composition or a mixture preferably occurs systematically, for example, by intravenous or intraperitoneal administration. In a further embodiment, such treatment also comprises the use of, for example, an antibiotic, however, only when such use is not contra indicated because of the risk of generating further toxin loads due to lysis of the bacteria by those antibiotics.

The invention also provides use of a composition according to the invention for the preparation of a pharmaceutical composition or medicament and methods of treating various medical conditions, such as an immune-mediated disorder.

### DETAILED DESCRIPTION OF THE INVENTION

As used herein, a “purified or isolated” peptide is one that has been purified from a natural or biotechnological source, or, more preferably, is synthesized as described herein.

“Composition,” as used herein, refers to chemical compounds which contain or consist of the peptide. The peptide is preferably isolated before inclusion within the composition. The peptide most preferably consists of three (3) to nine (9) amino acids.

As used herein, a “functional analogue” or “derivative” of a peptide includes variations made with regard to a reference peptide, which retains an identifiable relationship to the reference peptide, including variations made by pepscan, ala-scanning, replacement net analysis, methods disclosed in U.S. patent application Ser. No. 10/456,375 and/or conservative substitutions relative to the reference sequence, for example, SEQ ID NO:44. Derivatives also include compounds having the same or equivalent sidechains as the particular amino acids used in a peptide, and arranged sequentially in the same order as the peptides, but joined together by non-peptide bonds, e.g., by isosteric linkages such as the keto isostere, hydroxy isostere, diketo isostere, or the keto-difluoromethylene isostere. Once a derivative is produced, such a derivative is a peptide for the purposes of screening, identification of activity, inclusion in a database, production of a pharmaceutical and the like.

Also included within derivatives or functional analogues are peptidomimetic compounds that functionally or structurally resemble the original peptide taken as the starting point, but that are, for example, composed of non-naturally occurring amino acids or polyamides. With “conservative amino acid substitution”, one amino acid residue is substituted with another residue with generally similar properties (size, hydrophobicity, or charge), such that the overall functioning of a peptide sequence having such substitution is likely not to be seriously affected. A derivative can also be provided by systematically altering at least one amino acid of the reference peptide. This can, for instance, be done by an Alanine scanning (Ala-scan) and/or replacement net analysis, in which each amino acid is replaced in turn with one of the 19 (or 21, if selenocysteine and pyrrolysine are included) other amino acids. With these methods, many different peptides may be generated, based on an original amino acid sequence but each containing a variation or substitution of at least one amino acid residue. This way, many positional variants of the original amino acid sequence are synthesized and/or enzymatically prepared.

A derivative or analogue can also be, for instance, generated by substitution of an L-amino acid residue with a D-amino acid residue. Such a substitution may improve a property of an amino acid sequence, for example, to provide a peptide sequence of known activity of all D-amino acids in retro inversion format, thereby allowing for retained activity and increased half-life values. By generating many positional variants (derivatives) of an original amino acid sequence and screening for a specific activity, an improved peptide, for example, comprising D-amino acids, can be identified and used according to the invention.

The peptides (which include derivatives and/or functional analogues) may optionally be arranged on a chip, array or in a multiwell format (for array technology see, U.S. Pat. Nos. 6,630,308; 6,610,482; 6,506,558; 6,346,413; and 6,329,143). The peptides may be screened for a specific activity. Optionally, the generated data may further be used to design improved peptide derivatives of a certain amino acid sequence.

For instance, the previously described preferred compound could, in one embodiment be:
NT Xaa₁ Xaa₂ Xaa₃ Xaa₄ CT
wherein NT at the N-terminus is selected from the group of H—, CH₃—, an acyl group, or a general protective group; and CT at the C-terminus is selected from the group of small (e.g., 1 to 5 amino acids) peptides, —OH, —OR¹, —NH₂, —NHR¹, —NR¹R², or —N(CH₂)₁₋₆ NR¹R², wherein R¹ and R², when present, are independently selected from H, alkyl, aryl, (ar)alkyl, and wherein R¹ and R² can be cyclically bonded to one another. Such modifications constitute derivatives of the reference peptide.

“Alkyl” as used herein, is preferably a saturated branched or unbranched hydrocarbon having one to six carbon atoms, e.g., methyl, ethyl, and isopentyl.

“Aryl” as used herein, is an aromatic hydrocarbon group, preferably having 6 to 10 carbon atoms, such as phenyl or naphthyl.

“(Ar)alkyl”, as used herein, is an arene group (having both aliphatic and aromatic portions), preferably having 7 to 13 carbon atoms such as benzyl, ethylbenzyl, n-propylbenzyl, and isobutylbenzyl.

“Peptide,” as used herein, means peptides having from 2 to about 50 amino acids joined together by peptide bonds.

“Composition” also includes, for example, an acceptable salt of the oligopeptide or a labeled peptide. As used herein, “acceptable salt” refers to salts that retain the desired activity of the peptide or equivalent compound, but preferably do not detrimentally affect the activity of the peptide or other component of a system, which uses the peptide. Examples of such salts are acid addition salts formed with inorganic acids, for example, hydrochloric acid, hydrobromic acid, sulfuric acid, phosphoric acid, nitric acid, and the like. Salts may also be formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, pamoic acid, alginic acid, polyglutamic acid, and the like. Salts may be formed with polyvalent metal cations such as zinc, calcium, bismuth, barium, magnesium, aluminum, copper, cobalt, nickel and the like or with an organic cation formed from N,N′-dibenzylethylenediamine or ethylenediamine, or combinations thereof (e.g., a zinc tannate salt).

The composition can be administered or introduced in-vivo systemically, topically, or locally. The composition can be administered as a pharmaceutically acceptable acid- or base-addition salt, formed by reaction with an inorganic acid (such as hydrochloric acid, hydrobromic acid, perchloric acid, nitric acid, thiocyanic acid, sulfuric acid, and phosphoric acid); or with an organic acid (such as formic acid, acetic acid, propionic acid, glycolic acid, lactic acid, pyruvic acid, oxahc acid, malonic acid, succinic acid, maleic acid, and fumaric acid); or by reaction with an inorganic base (such as sodium hydroxide, ammonium hydroxide, potassium hydroxide); or with an organic base (such as mono-, di-, trialkyl and aryl amines and substituted ethanolamines). A peptide may also be conjugated to sugars, lipids, other polypeptides, nucleic acids and PNA; and function in situ as a conjugate or be released locally after reaching a targeted tissue or organ.

The compounds according to the invention may be prepared by methods known in the art (for example, see, U.S. patent application Ser. No. 10/456,375). For example, by peptide synthesis methods known in the art, including, suitable N alpha protection (and side-chain protection if reactive side-chains are present). The amino acid derivatives or peptides are activated and coupled to suitably carboxylprotected amino acid or peptide derivatives either in solution or on a solid support. Protection of the α-amino group may utilize an acid-labile tertiary-butyloxycarbonyl group (“Boc”), benzyloxycarbonyl (“Z”) group or substituted analogs or the base-labile 9-fluoremyl-methyloxycarbonyl (“Fmoc”) group. The Z group can also be removed by catalytic hydrogenation, other suitable protecting groups include Nps, Bmv, Bpoc, Aloc, MSC, etc. A good overview of amino protecting groups is given in The peptides, Analysis, Synthesis, Biology, Vol. 3 E. Gross and J. Meienhofer, eds., (Academic Press, New York, 1981). Protection of carboxyl groups can take place by ester formation, for example, base-labile esters like methyl or ethyl, acid labile esters like tertiary butyl or, substituted, benzyl esters or hydrogenolytically. Protection of side-chain functions like those of lysine and glutamic or aspartic acid can take place using the aforementioned groups. Protection of thiol, and although not always required, of guanidino, alcohol and imidazole groups can take place using a variety of reagents such as those described in The Peptides, Analysis, Synthesis, Biology, or in Pure and Applied Chemistry, 59(3), 331-344 (1987). Activation of the carboxyl group of the suitably protected amino acids or peptides can take place by the azide, mixed anhydride, active ester, or carbodiimide method especially with the addition of catalytic and racemization-suppressing compounds like 1-N-N-hydroxybenzotriazole, N-hydroxysuccin-imide, 3-hydroxy-4-oxo-3,4-dihydro-1,2,3,-benzotriazine, N-hydroxy-5 norbornene-2,3-dicarboxyimide. Also the anhydrides of phosphorus based acids can be used. See, e.g., The Peptides, Analysis, Synthesis, Biology, supra and Pure and Applied Chemistry, 59(3),331-344 (1987).

It is also possible to prepare the compounds by the solid phase method of Merrifield. Different solid supports and different strategies are known see, e.g. Barany and Merrifield in The Peptides, Analysis, Synthesis, Biology, Vol. 2, E. Gross and J. Meienhofer, eds., (Acad. Press, New York, 1980), Kneib-Cordonier and Mullen Int. J. Peptide Protein Res., 30, 705-739 (1987) and Fields and Noble Int. J. Peptide Protein Res., 35, 161-214 (1990). The synthesis of compounds in which a peptide bond is replaced by an isostere, can, in general, be performed using the previously described protecting groups and activation procedures. Procedures to synthesize the modified isosteres are described in the literature e.g., for the—CH₂—NH—isostere and for the—CO—CH₂—isostere.

Removal of the protecting groups, and, in the case of solid phase peptide synthesis, the cleavage from the solid support may be performed by means known in the art (see, e.g. volumes 3, 5 and 9 of the series on The Peptides Analysis, Synthesis, Biology, supra).

Another possibility is the application of enzymes in synthesis of such compounds; for reviews see, e.g., H. D. Jakubke in The Peptides, Analysis, Synthesis, Biology, Vol. 9, S. Udenfriend and J. Meienhofer, eds., (Acad. Press, New York, 1987). For example, by modifications such as glycosylation, phosphorylation and other modifications known in the art.

Peptides according to the invention may also be made according to recombinant DNA methods. Such methods involve the preparation of the desired peptide by means of expressing recombinant polynucleotide sequence which codes for one or more of the oligopeptides in a suitable host cell. Generally the process involves introducing into a cloning vehicle (e.g., a plasmid, phage DNA, or other DNA sequence able to replicate in a host cell) a DNA sequence coding for the particular oligopeptide or oligopeptides, introducing the cloning vehicle into a suitable eucaryotic or procaryotic host cell, and culturing the host cell thus transformed. When a eucaryotic host cell is used, the compound may include a glycoprotein portion.

The administration dose of the active molecule may be varied over a fairly broad range. The concentrations of an active molecule which can be administered would be limited by efficacy at the lower end and the solubility of the compound at the upper end. The optimal dose or doses for a particular patient should and can be determined by the physician or medical specialist involved, taking into consideration well-known relevant factors such as the condition, weight and age of the patient, etc.

The invention provides methods for screening and organizing the results from such screens, for example, regarding peptides derived from MTRVLQGVLPALPQVVC (SEQ ID NO:44) having a length of 3, 4, 5, 6, 7, 8, or 9 amino acids. In one exemplary embodiment, a scanning concept may utilize the process of looking at the linear protein sequence through a series of moving windows of a predetermined size. By way of example, peptides of four amino acid lengths (e.g., MTRV, TRVL, RVLQ, VLQG, LQGV, QGVL, GVLP, VLPA, LPAL, PALP, etc. through QVVC (SEQ ID NOs: 176-179, 1, 180-185, respectively)) are produced from MTRVLQGVLPALPQVVC (SEQ ID NO:44). In another embodiment, derivatives of one or more tetramer are produced by an amino acid variation procedure, such as a replacement net analysis or other methods known in the art (see, for example, Ward et al. (1996) Systematic Mapping of Potential Binding Sites for Shc and Grb2 SH2 Domains on Insulin Receptor Substrate-1 and the Receptors for Insulin, Epidermal Growth Factor, Platelet-derived Growth Factor, and Fibroblast Growth Factor, J. Biol. Chem. 271(10):5603-5609), thereby producing a library of tetramers that may be screened for useful characteristics, such as modulation of sepsis and/or the prevention or treatment of diabetes.

The invention alsom provides methods for screening, organizing and using the results from such screens, for example, with peptides derived from a refernce peptide. For example, peptides to be analyzed in this way may be derived from the reference peptide C-Reactive Protein (CRP) (ie.g., human CRP), such peptides include, LTSL, FVLS, NMWD, LCFL, MWDF, FSYA, FWVD, AFTV, and WDFV (SEQ ID NOs:186-194, respectively); peptides derived from Beta-catenin (e.g., human CTNB), such as GLLG, TAPS, VCQV, CLWT, VHQL, GALH, LGTL, TLVQ, QLLG, YAIT, LCEL, GLIR, APSL, ITTL, QALG, HPPS, GVLC, LCPA, LFYA, NIMR, NLIN, LHPP, LTEL, SPIE, VGGI, QLLY, LNTI, LWTL, LYSP, YAMT, LHNL, TVLR, and LFYA (SEQ ID NOs:195-227, respectively); peptides derived from beta-hCG (e.g., human CGHB), such as GLLLLLLLS, MGGTWA, TWAS, TLAVE, RVLQ, VCNYRDV, FESI, RLPG, PRGV, NPVVS, YAVALS, LTCDDP, EMFQ, PVVS, VSYA, GVLP, FQGL, and AVAL (SEQ ID NOs:228-245, respectively); peptides derived from Bruton's tyrosine kinase (e.g., human BTK), such as LSNI, YVFS, LYGV, YVVC, FIVR, NILD, TIMY, LESI, FLLT, VFSP, FILE, TFLK, FWID, MWEI, QLLE, PCFW, VHKL, LYGV, LESI, LSNI, YVFS, IYSL, and NILD (SEQ ID NOs:246-268, respectively); and peptides derived from matrix metalloproteinase-2 (e.g., human MM02), such as FKGA, FFGL, GIAQ, LGCL, YWIY, AWNA, ARGA, PFRF, APSP CLLS, GLPQ, TFWP, AYYL, FWPE, CLLG, FLWC, RIIG, WSDV, PIIK, GLPP, RALC, LNTF, LSHA, ATFW, PSPI, AHEF, WRTV, FVLK, VQYL, KFFG, FPFR, IYSA, and FDGI (SEQ ID NOs:269-301, respectively).

A non-extensive list of relevant oligopeptides useful for application in a method and/or database according to the invention include:
Accession number pdb|1DE7|1DE7-A, see INTERACTION OF FACTOR XIII ACTIVATION PEPTIDE WITH ALPHA-THROMBIN, J. Biol. Chem. 2000 275(47):36942-8, which includes LQGV (SEQ ID NO:1), LQGVV (SEQ ID NO:53), and LQGVVP (SEQ ID NO:54);
Accession number pdb|1DL6|1DL6-A (see also Accession number Q00403) SOLUTION STRUCTURE OF HUMAN TFIIB N-TERMINAL DOMAIN, including LDALP (SEQ ID NO:55);
Accession number pdb|1QMH|1QMH-A, see CRYSTAL STRUCTURE OF RNA 3′-TERMINAL PHOSPHATE CYCLASE, AN UBIQUITOUS ENZYME (accession number NP_{—}709195), which includes LQTV (SEQ ID NO:56), VLPAL (SEQ ID NO:8), and LVLQTVLPAL (SEQ ID NO:57);
Accession number pdb|1LYP|1LYP, CAP18 (RESIDUES 106-137), which includes IQG, IQGL (SEQ ID NO:58), LPKL (SEQ ID NO:59), and LLPKL (SEQ ID NO:60);
Accession number pdb|1B9O|1B9O-A HUMAN ALPHA-LACTALBUMIN (accession number P00709), which includes LPEL (SEQ ID NO:61);
Accession number pdb|1GLU|1GLU-A GLUCOCORTICOID RECEPTOR (DNA-BINDING DOMAIN) (accession number PO₆₅₃₆), which includes PARP (SEQ ID NO:62);
Accession number pdb|2KIN|2KIN-B KINESIN (MONOMERIC) FROM RATTUS NORVEGICUS (see also, accession number P56536), which includes MTR1 (SEQ ID NO:63);
Accession number pdb|1SMP|1SMP-I MOL_ID: 1; MOLECULE: SERRATIA METALLO PROTEINASE; CHAIN: A (see also, accession number P18958), which includes LQKL (SEQ ID NO:64), LQKLL (SEQ ID NO:65), PEAP (SEQ ID NO:66), and LQKLLPEAP (SEQ ID NO:67);
Accession number pdb|1ES7|1ES7-B COMPLEX BETWEEN BMP-2 AND TWO BMP RECEPTOR IA ECTODOMAINS (see also, accession numbers P36894 and P12643), which includes LPQ, PTLP (SEQ ID NO:68), and LQPTL (SEQ ID NO:69);
Accession number pdb|1BHX|1BHX-F X-RAY STRUCTURE OF THE COMPLEX OF HUMAN ALPHA THROMBIN WITH THE INHIBITOR SDZ 229-357 (see also, accession number P00734),. which includes LQV, and LQVV (SEQ ID NO:70);
Accession number pdb|1VCB|1VCB-A THE VHL-ELONGINC-ELONGINB STRUCTURE (see also, accession number BI067547 for the nucleotide sequence), which includes PELP (SEQ ID NO:71);
Accession number pdb|1CQK|1CQK-A CRYSTAL STRUCTURE OF THE CH₃ DOMAIN FROM THE MAK33 ANTIBODY (see also, accession number 1CQKB), which includes PAAP (SEQ ID NO:72), PAAPQ (SEQ ID NO:73), and PAAPQV (SEQ ID NO:74);
Accession number pdb|1FCB|1FCB-A FLAVOCYTOCHROME (see also, accession number P00175), which includes LQG;
Accession number pdb|1LDC|1LDC-A L-LACTATE DEHYDROGENASE: CYTOCHROME C OXIDOREDUCTASE (FLAVOCYTOCHROME B=2=) (E.C.1.1.2.3) MUTANT WITH TYR 143 REPLACED BY PHE (Y143F) COMPLEXED WITH PYRUVATE (see also, accession number P00175), which includes LQG;
Accession number pdb|1BFB|1BFB BASIC FIBROBLAST GROWTH FACTOR COMPLEXED WITH HEPARIN TETRAMER FRAGMENT, which includes LPAL (SEQ ID NO:183), PALP (SEQ ID NO:184), and PALPE (SEQ ID NO:77);
Accession number pdb|1MBF|1MBF MOUSE C-MYB DNA-BINDING DOMAIN REPEAT 1, which includes LPN;
Accession number pdb|1R2A|1R2A-A THE MOLECULAR BASIS FOR PROTEIN KINASE A (see also, accession number P12367), which includes LQG, and LTELL (SEQ ID NO:78);
Accession number pdb|1CKA|1CKA-B C—CRK (N-TERMINAL SH3 DOMAIN) (C—CRKSH3—N)COMPLEXED WITH C3G PEPTIDE (PRO-PRO-PRO-ALA-LEU-PRO-PRO-LYS-LYS-ARG) (see also, accession number Q64010), which includes PALP (SEQ ID NO:184);
Accession number pdb|1RLQ|1RLQ-R C—SRC(SH3 DOMAIN) COMPLEXED WITH THE PROLINE-RICH LIGAND RLP2 (RALPPLPRY) (NMR, MINIMIZED AVERAGE STRUCTURE) (see also, accession number P00523), which includes LPPL (SEQ ID NO:80), and PPLP (SEQ ID NO:81);
Accession number pdb|1TNT|1TNT MU TRANSPOSASE (DNA-BINDING DOMAIN) (NMR, 33 STRUCTURES) (see also, accession number GI:999952), which includes LPG, LPGL (SEQ ID NO:82), and LPK;
Accession number pdb|1GJS|1GJS-A SOLUTION STRUCTURE OF THE ALBUMIN BINDING DOMAIN OF STREPTOCOCCAL PROTEIN G (see also, accession number P19909), which includes LAAL (SEQ ID NO:83), and LAALP (SEQ ID NO:84);
Accession number pdb|1GBR|1GBR-B GROWTH FACTOR RECEPTOR-BOUND PROTEIN 2 (GRB2, N-TERMINAL SH3 DOMAIN) COMPLEXED WITH SOS-A PEPTIDE (NMR, 29 STRUCTURES) (see also, accession number Q60631), which includes LPKL (SEQ ID NO:59), and PKLP(SEQ ID NO:85);
Accession number pdb|1A78|1A78-A COMPLEX OF TOAD OVARY GALECTIN WITH THIO-DIGALACTOSE (see also, accession number P56217), which includes VLPSIP (SEQ ID NO:86);
Accession number pdb|1ISA|1ISA-A IRON(II) SUPEROXIDE DISMUTASE (E.C.1.15.1.1) (see also, accession number P09157), which includes LPAL (SEQ ID NO:183), and PALP (SEQ ID NO:184);
Accession number pdb|1FZV|1FZV-A THE CRYSTAL STRUCTURE OF HUMAN PLACENTA GROWTH FACTOR-1 (PLGF-1), AN ANGIOGENIC PROTEIN AT 2.0A RESOLUTION (see also, accession number P49763), which includes PAVP (SEQ ID NO:311), and MLPAVP (SEQ ID NO:87);
Accession number pdb|1JLI|1JLI HUMAN INTERLEUKIN 3 (IL-3) MUTANT WITH TRUNCATION AT BOTH N— AND C-TERMINI AND 14 RESIDUE CHANGES, NMR, MINIMIZED AVERAGE (see also, accession number GI:2392392), which includes LPC, LPCL (SEQ ID NO:88), and PCLP (SEQ ID NO:89);
Accession number pdb|1HSS|1HSS-A 0.19 ALPHA-AMYLASE INHIBITOR FROM WHEAT (see also, accession number P01085), which includes VPALP (SEQ ID NO:90);
Accession number pdb|3CRX|3CRX-A CRE RECOMBINASE/DNA COMPLEX INTERMEDIATE I (see also, accession number P06956), which includes LPA, LPAL (SEQ ID NO:183), and PALP (SEQ ID NO:184);
Accession number pdb|1PRX|1PRX-A HORF6 A NOVEL HUMAN PEROXIDASE ENZYME (see also, accession number P30041), which includes PTIP (SEQ ID NO:91), and VLPTIP (SEQ ID NO:92);
Accession number pdb|1RCY|1RCY RUSTICYANIN (RC) FROM THIOBACILLUS FERROOXIDANS (see also, accession number GI:2194027), which includes VLPGFP (SEQ ID NO:93);
Accession number pdb|1A3Z|1A3Z REDUCED RUSTICYANIN AT 1.9 ANGSTROMS (see also, accession number GI:3402027), which includes PGFP (SEQ ID NO:94), and VLPGFP (SEQ ID NO:93);
Accession number pdb|1GER|1GER-A GLUTATHIONE REDUCTASE (E.C.1.6.4.2) COMPLEXED WITH FAD (see also, accession number PO₆₇₁₅), which includes LPALP (SEQ ID NO:95), and PALP (SEQ ID NO:184);
Accession number pdb|1PBW|1PBW-A STRUCTURE OF BCR—HOMOLOGY (BH) DOMAIN (see also, accession number P27986), which includes PALP (SEQ ID NO:184);
Accession number pdb|1BBS|1BBS RENIN (E.C.3.4.23.15), which includes MPALP (SEQ ID NO:96);
Accession number AI188872 11.3 366 327 18 382 [Homo sapiens]qd27c01.x1 Soares_placenta_{—}8 to 9 weeks_{—}2NbHP8 to 9W, Homo sapiens cDNA clone IMAGE:1724928 3′ similar to gb:J00117 CHORIOGONADOTROPIN BETA CHAIN PRECURSOR (HUMAN);, mRNA sequence.; minus strand; translated, which includes MXRVLQGVLPALPQVVC (SEQ ID NO:97), MXRV (SEQ ID NO:98), and MXR;
Accession number AI126906 19.8 418 343 1 418 [Homo sapiens]qb95f01.xl Soares_fetal_heart_NbHH19W Homo sapiens cDNA clone IMAGE:1707865 3′ similar to gb:J00117 CHORIOGONADOTROPIN BETA CHAIN PRECURSOR (HUMAN);, mRNA sequence.; minus strand; translated, which includes ITRVMQGVIPALPQVVC (SEQ ID NO:99);
Accession number AI221581 29.1 456 341 23 510 [Homo sapiens]qg20a03.xl Soares placenta 8 to 9 weeks 2NbHP8 to 9W Homo sapiens cDNA clone IMAGE: 1760044 3′ similar to gb:J00117 CHORIOGONADOTROPIN BETA CHAIN PRECURSOR (HUMAN);, mRNA sequence.; minus strand; translated, which includes MTRVLQVVLLALPQLV (SEQ ID NO:100);
Accession number Mm.42246.3 Mm.42246 101.3 837 304 28 768 GENE=Pckl PROTSIM=pir:T24168 phosphoenolpyruvate carboxykinase 1, cytosolic; translated, which includes KVIQGSLDSLPQAV (SEQ ID NO:101), LDSL (SEQ ID NO:102), and LPQ;
Accession number Mm.22430.1 Mm.22430 209.4 1275 157 75 1535 GENE=Ask-pending PROTSIM=pir:T02633 activator of S phase kinase; translated, which includes VLQAILPSAPQ (SEQ ID NO:103), LQA, LQAIL (SEQ ID NO:104), PSAP (SEQ ID NO:105), and LPS;
Accession number Hs.63758.4 Hs.63758 93.8 3092 1210 51 2719 GENE=TFR2 PROTSIM=pir:T30154 transferrin receptor 2; translated, which includes KVLQGRLPAVAQAV (SEQ ID NO:106), LQG, LPA, and LPAV (SEQ ID NO:107);
Accession number Mm.129320.2 Mm.129320 173.0 3220 571 55 2769 GENE=PROTSIM=pir:T16409 Sequence 8 from Patent WO9950284; translated, which includes LVQKVVPMLPRLLC (SEQ ID NO:108), LVQ, LPRL (SEQ ID NO:109), and PMLP (SEQ ID NO:110);
Accession number Mm.22430.1 Mm.22430 209.4 1275 157 75 1535 GENE=Ask-pending PROTSIM=pir:T02633 activator of S phase kinase; translated, which includes VLQAILPSAPQ (SEQ ID NO:103), LQA, LQAIL (SEQ ID NO:104), PSAP (SEQ ID NO:105), and PSAPQ (SEQ ID NO:111);
Accession number P20155.1 AC2_HUMAN Acrosin-trypsin inhibitor II precursor (HUSI-II) [SPINK2] [Homo sapiens], which includes LPGCPRHFNPV (SEQ ID NO:112), LPG, and LPGC (SEQ ID NO:41);
Accession number Rn.2337.1 Rn.2337 113.0 322 104 1 327 GENE=PROTSIM=PRF:1402234A Rat pancreatic secretory trypsin inhibitor type II (PSTI-II) mRNA, complete cds; minus strand; translated, which includes LVGCPRDYDPV (SEQ ID NO:113), LVG, and LVGC (SEQ ID NO:114);
Accession number Hs.297775.1 Hs.297775 43.8 1167 753 31 1291 GENE=PROTSIM=sp:000268 ESTs, Weakly similar to T2D3_HUMAN TRANSCRIPTION INITIATION FACTOR TFIID 135 KDA SUBUNIT [H.sapiens]; minus strand; translated, which includes PGCPRG (SEQ ID NO:115), and PGCP (SEQ ID NO:10);
Accession number Mm.1359.1 Mm.1359 PROTSTM=pir.A39743 urokinase plasmiogen activator receptor, which includes LPGCP (SEQ ID NO: 116), PGCP (SEQ ID NO: 10), LPG, and LPGC (SEQ ID NO:41);
Accession number sptremb1|O56177|O56177 ENVELOPE GLYCOPROTEIN, which includes VLPAAP (SEQ ID NO: 117), and PAAP (SEQ ID NO:72);
Accession number sptremb1|Q9W234|Q9W234 CG13509 PROTEIN.//:trembl|AE003458|AE003458_{—}7 gene: “CG13509”; Drosophila melanogaster genomic scaffold, which includes LAGTIPATP (SEQ ID NO:118), LAG, and PATP (SEQ ID NO:119);
Accession number swiss|P81272|NS2B_HUMAN NITRIC-OXIDE SYNTHASE IIB (EC 1.14.13.39) (NOS, TYPE II B) (NOSIIB) (FRAGMENTS), which includes GVLPAVP, LPA, VLPAVP (SEQ ID NO:12), and PAVP (SEQ ID NO:31 1);
Accession number sptremb1|O30137|O30137 HYPOTHETICAL 17.2 KDA, which includes GVLPALP (SEQ ID NO:32), PALP (SEQ ID NO: 184), and LPAL (SEQ ID NO: 183);
Accession number sptremb1|Q91YZ3|Q91YZ3 DNA POLYMERASE, which includes GLLPCLP (SEQ ID NO:120), LPC, LPCL (SEQ ID NO:88), and PCLP (SEQ ID NO:89);
Accession number sptremb1|Q9PVW5|Q9PVW5 NUCLEAR PROTEIN NP220, which includes PGAP (SEQ ID NO:121), LPQRPRGPNP (SEQ ID NO:122), LPQ, PRGP (SEQ ID NO:123), and PNP;
Accession number Hs.303116.2 PROTSIM=pir;T33097 stromal cell-derived factor 2-like1; translated, which includes GCPR (SEQ ID NO: 124);
Accession number pdb|1DU3|1DU3-A CRYSTAL STRUCTURE OF TRAIL-SDR5, which includes GCPRGM (SEQ ID NO: 125);
Accession number pdb|1D0G|1DOG-R CRYSTAL STRUCTURE OF DEATH RECEPTOR 5 (DR5) BOUND TO APO2L/TRAIL, which includes GCPRGM (SEQ ID NO: 125);
Accession number pdb|1BIO|1BIO HUMAN COMPLEMENT FACTOR D IN COMPLEX WITH ISATOIC ANHYDRIDE INHIBITOR, which includes LQHV (SEQ ID NO: 126);
Accession number pdb|4NOS|4NOS-A HUMAN INDUCIBLE NITRIC OXIDE SYNTHASE WITH INHIBITOR, which includes FPGC (SEQ ID NO:9), and PGCP (SEQ ID NO:10);
Accession number pdb|1FL7|1FL7-B HUMAN FOLLICLE STIMULATING HORMONE, which includes PARP (SEQ ID NO:62), and VPGC (SEQ ID NO:127);
Accession number pdb|1HR6|1HR6-A YEAST MITOCHONDRIAL PROCESSING PEPTIDASE, which includes CPRG (SEQ ID NO: 128), and LKGC (SEQ ID NO: 129);
Accession number pdb|1BFA|1BFA RECOMBINANT BIFUNCTIONAL HAGEMAN FACTOR/AMYLASE INHIBITOR FROM, which includes PPGP (SEQ ID NO:130), LPGCPREV (SEQ ID NO:131), LPGC (SEQ ID NO:41), PGCP (SEQ ID NO:10), and CPRE (SEQ ID NO:132);
Accession number swissnew|P01229|LSHB_HUMAN Lutropin beta chain precursor, which includes MMRVLQAVLPPLPQVVC (SEQ ID NO:133), MMR, MMRV (SEQ ID NO:383), LQA, LQAV (SEQ ID NO:52), VLPPLP (SEQ ID NO:135), PPLP (SEQ ID NO:82), QVVC (SEQ ID NO:43), VVC, VLPPLPQ (SEQ ID NO:136), AVLPPLP (SEQ ID NO:137), and AVLPPLPQ (SEQ ID NO:138);
Accession number swissnew|P07434|CGHB_PAPAN Choriogonadotropin beta chain precursor, which includes MMRVLQAVLPPVPQVVC (SEQ ID NO:312), MMR, MMRV (SEQ ID NO:134), LQA, LQAG (SEQ ID NO:140), VLPPVP (SEQ ID NO:141), VLPPVPQ (SEQ ID NO:142), QVVC (SEQ ID NO:43), VVC, AVLPPVP (SEQ ID NO:143), and AVLPPVPQ (SEQ ID NO: 144);
Accession number swissnew|Q283761TSHB_HORSE Thyrotropin beta chain precursor, which includes MTRD (SEQ ID NO:145), LPK, QDVC (SEQ ID NO:146), DVC, IPGC (SEQ ID NO:147), and PGCP (SEQ ID NO:10);
Accession number swissnew|P95180|NUOB_MYCTU NADH dehydrogenase I chain B, which includes LPGC (SEQ ID NO:41), and PGCP (SEQ ID NO:10); Accession number sptremb1|Q9Z2841Q9Z284 NEUTROPHIL ELASTASE, which includes PALP (SEQ ID NO:184), and PALPS (SEQ ID NO:148);
Accession number sptremb1|Q9UCG81Q9UCG8 URINARY GONADOTROPHIN PEPTIDE (FRAGMENT), which includes LPGGPR (SEQ ID NO:149), LPG, LPGG (SEQ ID NO:150), and GGPR (SEQ ID NO:151); and
Accession number XP_{—}028754 growth hormone releasing hormone [Homo sapiens], which includes LQRG (SEQ ID NO:152), LQRGV (SEQ ID NO:153), and LGQL (SEQ ID NO: 154). All of which are hereby incorporated in their entirety by reference.

A further non-limiting list includes collagen, PSG, CEA, MAGE (malanoma associated growth antigen), Thrombospondin-1, Growth factors, MMPs, Calmodulin, Olfactory receptors, Cytochrome p450, Kinases, Von Willebrand factor (coagulation factors), Vacuolar proteins (ATP sythase), Glycoprotein hormones, DNA polymerase, Dehydrogenases, Amino peptidases, Trypsin, Viral proteins (such as envelope protein), Elastin, Hibernation associated protein, Antifreeze glycoprotein, Proteases, Circumsporozoite, Nuclear receptors, Transcription actors, Cytokines and their receptors, Bacterial antigens, Nramp, RNA polymerase, Cytoskeletal proteins, Hematopoietic (neural) membrane proteins, Immunoglobulins. HLA/MHC, G-coupled proteins and their receptors, TATA binding proteins, Transferases, Zinc finger protein, Spliceosmal proteins, HMG (high mobility group protein), ROS (reactive oxygen species), superoxidases, superoxide dismutase, Proto-oncogenes/tumor suppressor genes, and Apolipoproteins.

The invention is further explained with the aid of the following illustrative examples.

### EXAMPLES

### Example I

Material and Methods

PEPTIDE SYNTHESIS: The peptides as mentioned herein such as LQG, AQG, LQGV (SEQ ID NO:1), AQGV (SEQ ID NO:2), LQGA (SEQ ID NO:19), VLPALP (SEQ ID NO:13), ALPALP (SEQ ID NO:21), VAPALP (SEQ ID NO:22), ALPALPQ (SEQ ID NO:23), VLPAAPQ (SEQ ID NO:24), VLPALAQ (SEQ ID NO:25), LAGV (SEQ ID NO:26), VLAALP (SEQ ID NO:27), VLPALA (SEQ ID NO:28), VLPALPQ (SEQ ID NO:29), VLAALPQ (SEQ ID NO:30), VLPALPA (SEQ ID NO:31), GVLPALP (SEQ ID NO:32), VVCNYRDVRFESIRLPGCPRGVNPVVSYAVALSCQCAL (SEQ ID NO:35), RPRCRPINATLAVEKEGCPVCITVNTTICAGYCPT (SEQ ID NO:45), SKAPPPSLPSPSRLPGPS (SEQ ID NO:38), LQGVLPALPQVVC (SEQ ID NO:34), SIRLPGCPRGVNPVVS (SEQ ID NO:39), LPGCPRGVNPVVS (SEQ ID NO:40), LPGC (SEQ ID NO:41), MTRV (SEQ ID NO:42), MTR, and VVC were prepared by solid-phase synthesis (R. B. Merrifield, J. Am. Chem. Soc., 85:2149-2165 (1963)) using the fluorenylmethoxycarbonyl (Fmoc)/tert-butyl-based methodology (Atherton, 1985) with 2-chlorotrityl chloride resin (Barlos et al., Int. J. Peptide Protein res., 37:513-520 (1991)) as the solid support.

The side-chain of glutamine was protected with a trityl function. The peptides were synthesized manually. Each coupling consisted of the following steps: (i) removal of the alpha-amino Fmoc-protection by piperidine in dimethylformamide (DMF), (ii) coupling of the Fmoc amino acid (3 eq) with diisopropylcarbodiimide (DIC)/1-hydroxybenzotriazole (HOBt) in DMF/N-methylformamide (NMP) and (iii) capping of the remaining amino functions with acetic anhydride/diisopropylethylamine (DIEA) in DMF/NMP. Upon completion of the synthesis, the peptide resin was treated with a mixture of trifluoroacetic acid (TFA)/H₂O/triisopropylsilane (TIS) 95:2.5:2.5. After 30 minutes, TIS was added until decolorization. The solution was evaporated in vacuo and the peptide precipitated with diethylether.

The crude peptides were dissolved in water (50-100 mg/ml) and purified by reverse-phase high-performance liquid chromatography (RP-HPLC). HPLC conditions were: column: Vydac TP21810C18 (10×250 mm); elution system: gradient system of 0.1% TFA in water v/v (A) and 0.1% TFA in acetonitrile (ACN) v/v (B); flow rate 6 ml/min; absorbance was detected from 190-370 nm. There were different gradient systems used. For example, for peptides LQG and LQGV (SEQ ID NO:1): 10 minutes 100% A followed by linear gradient 0-10% B in 50 minutes. For example for peptides VLPALP (SEQ ID NO:3) and VLPALPQ (SEQ ID NO:29): 5 minutes 5% B followed by linear gradient 1% B/minute. The collected fractions were concentrated to about 5 ml by rotation film evaporation under reduced pressure at 40° C. The remaining TFA was exchanged against acetate by eluting two times over a column with anion exchange resin (Merck II) in acetate form. The eluate was concentrated and lyphilized in 28 hours. Peptides later were prepared for use by dissolving them in PBS.

### Example II

Endotoxin Shock Model (Sepsis)

Sepsis. For the endotoxin model, BALB/c mice were injected i.p. with 8-9 mg/kg LPS (E. coli 026:B6; Difco Lab., Detroit, Mich., USA). Control groups (PBS) were treated with PBS i.p. only. To test the effect of NMPF from different sources (synthetic, commercial hCG preparation [c-hCG]), we treated BALB/c mice with a dose of 300-700 IU of different hCG preparations (PG23; PREGNYL™ batch no. 235863, PG25; PREGNYL™ batch no. 255957 from NV Organon of Oss, NL) and with synthetic peptides (5 mg/kg) after two hours of LPS injection. In other experiments, BALB/c mice were injected i.p. either with 10 mg/kg or with 11 mg/kg LPS (E. coli 026:B6; Difco Lab., Detroit, Mich., USA). Subsequently, mice were treated after 2 hours and 24 hours of LPS treatment with NMPF peptides.

Semi-quantitative sickness measurements. Mice were scored for sickness severity using the following measurement scheme:
1 Percolated fur, but no detectable behaviour differences compared to normal mice.
2 Percolated fur, huddle reflex, responds to stimuli (such as tap on cage), just as active during handling as healthy mouse.
3 Slower response to tap on cage, passive or docile when handled, but still curious when alone in a new setting.
4 Lack of curiosity, little or no response to stimuli, quite immobile.
5 Labored breathing, inability or slow to self-right after being rolled onto back (moribund)
6 Sacrificed
   Results
   Endotoxin Shock Model (Sepsis)

Sepsis experiments. To determine the effect of synthetic peptides (NMPF) in high-dose LPS shock model, BALB/c mice were injected intraperitoneally with different doses of LPS and survival was assessed daily for 5 days. In this experiment (for the LPS endotoxin model), BALB/c mice were injected i.p. with 8-9 mg/kg LPS (E. coli 026:B6; Difco Lab., Detroit, Mich., USA). Control groups (PBS) were treated with PBS i.p. only. We treated BALB/c mice with a dose of 300-700 IU of different hCG preparations (PG23; PREGNYL batch no. 235863, PG25; PREGNYL batch no. 255957) or with peptides (5 mg/kg) after two hours of LPS injection.

These experiments showed (Table 1) that NMPF peptides 4, 6, 66 and PG23 inhibited shock completely (all mice had in first 24 hours sickness scores not higher than 2; shortly thereafter they recovered completely and had sickness scores of 0), while peptides 2, 3 and 7 accelerated shock (all mice had in first 24 hours sickness scores of 5 and most of them died, while the control mice treated with LPS+PBS had sickness scores of 3-4 in first 24 hours and most of them died after 48 hours with sickness scores of 5; 17% survival rate at 72 hours). In addition, peptides 1, 5, 8, 9, 11, 12, 13, 14 and 64 showed in a number of different experiments variability in effectiveness as well as in the kind (inhibitory vs accelerating) of activity. This variability is likely attributable to the rate of breakdown of the various peptides and the different effects the various peptides and their breakdown products have in vivo. In addition, these experiments also showed the variability in anti-shock activity in c-hCG preparations that is likely attributable to the variation in the presence of anti-shock and shock-accelerating NMPF. Visible signs of sickness were apparent in all of the experimental animals, but the kinetics and obviously the severity of this sickness were significantly different. These data are representative of at least 10 separate experiments.

In Table 2, we see the effect of ALA-replacement (PEPSCAN) in peptide LQG, LQGV (SEQ ID NO:1), VLPALP (SEQ ID NO:3), VLPALPQ (SEQ ID NO:29) in septic shock experiments. The change of even one amino acid by a neutral amino acid can lead to different activity. So, genomic differences as well as polymorphism in these peptides can regulate the immune response very precisely. Derivatives of these peptides, for example (but not limited to) by addition of classical and non-classical amino acids or derivatives that are differentially modified during or after synthesis, for example benzylation, amidation, glycosylation, proteolytic cleavage, linkage to an antibody molecule or other cellular ligand, etc. may be assayed to identify the activity of each peptide.

Assay results may be compiled in a database and, optionally, the results may be sorted, for example, by the kinetics of visible signs of sickness.

To determine whether treatment of BALB/c mice with NMPF inhibits septic shock at different stages of disease, synthetic peptides (NMPF) were injected i.p. at 2 and 24 hours after the induction of septic shock with high dose LPS (10 mg/kg).

As shown in Tables 3 and 4, control mice treated with PBS after the shock induction reached a sickness score of 5 at 14 and 24 hours, and remained so after the second injection with PBS. The survival rate in control group mice was 0% at 48 hours. In contrast to control mice, mice treated with NMPF 9, 11, 12, 43, 46, 50 and 60 reached a maximum sickness score of 2-3 at 24 hours after the induction of septic shock and further reached a maximum sickness score of 1-2 at 48 hours after the second injection of NMPF. In addition, mice treated with NMPF 5, 7, 8, 45, 53 and 58 reached a sickness score of 5 and after the second injection with NMPF all mice returned to a sickness score of 1-2 and survival rates in NMPF groups were 100%. Mice treated with NMPF 3 reached sickness scores of 3-4 and the second NMPF injection did save these mice. These experiments show that NMPF peptides have anti-shock activity at different stages of the disease and NMPF have anti-shock activity even at the disease stage when otherwise irreversible damage had been done. This indicates that NMPF have effects on different cellular levels and also have repairing and regenerating capacity.

### Example III

NOD Experiment

Mice. Female NOD mice at the age of 13-14 weeks were treated i.p. with PBS (n=6) or NMPF peptides (VLPALPQVVC (SEQ ID NO:20), LQGV (SEQ ID NO:1), GVLPALPQ (SEQ ID NO:33), VLPALP (SEQ ID NO:3), VLPALPQ (SEQ ID NO:29), MTRV (SEQ ID NO:42), LPGCPRGVNPVVS (SEQ ID NO:40), CPRGVNPVVS (SEQ ID NO:50), LPGC (SEQ ID NO:41), MTRVLQGVLPALPQVVC (SEQ ID NO:44), VVCNYRDVRFESIRLPGCPRGVNPVVSYAVALSCQCAL (SEQ ID NO:35)) (5 mg/kg, n=6) three times a week for 2 weeks. Every four days urine was checked for the presence of glucose (Gluketur Test; Boehringer Mannheim, Mannheim, Del.). All mice used in these studies were maintained in a pathogen-free facility. They were given free access to food and water. The experiments were approved by the Animal Experiments Committee of the Erasmus University Rotterdam. Diabetes was assessed by measurement of the venous blood glucose level using an Abbott Medisense Precision glucometer. Mice were considered diabetic after two consecutive glucose measurements ≧11 mmol/l (200 mg/dl). Onset of diabetes was dated from the first consecutive reading.

Glucose tolerance test (GTT) was performed at 28 weeks of age in fasted mice (n=5) by injecting 1 g/kg D-glucose intraperitoneally (i.p.). At 0 (fasting), 5, 30 and 60 minutes, blood samples were collected from the tail and tested for glucose content.

### Example IV

NO Experiment

Cell culture. The RAW 264.7 murine macrophage cell line, obtained from American Type Culture Collection (Manassas, Va., USA), were cultured at 37° C. in 5% CO₂ using DMEM containing 10% fetal calf serum (FCS), 50 U/ml penicillin, 50 pg/ml streptomycin, 0.2 M Na-pyruvate, 2 mM glutamine and 50 μM 2-mercaptoethanol (Bio Whittaker, Europe). The medium was changed every 2 days.

Nitrite measurements. Nitrite production was measured in the RAW 264.7 macrophage supernatants. The cells (7.5×10⁵/ml) were cultured in 48-well plates in 500 μl of culture medium. The cells were stimulated with LPS (10 microg/ml) and/or NMPF (1 μg/ml, 1 ng/ml, 1 μg/ml) for 24 hours, then the culture media were collected. Nitrite was measured by adding 100 microl of Griess reagent (Sigma) to 100 microl samples of culture medium. The OD₅₄₀ was measured using a microplate reader, and the nitrite concentration was calculated by comparison with the OD₅₄₀ produced using standard solutions of sodium nitrite in the culture medium.

Results

NOD Experiment

In order to determine whether NMPF has effect on the disease development in NOD mice, we tested NMPF on pre-diabetic female NOD mice at the age of 13-14 weeks. After only two weeks of treatment (injection of NMPF (5 mg/kg) every other day), glucosuria data of all NOD mice was analyzed at the of 17 weeks. Profound anti-diabetic effect (mice negative for glucosuria) was observed in different NMPF groups as compared to the PBS group, especially in NMPF groups treated with peptide VLPALPQVVC (SEQ ID NO:20), VLPALP (SEQ ID NO:3), MTRV (SEQ ID NO:42), LPGCPRGVNPVVS (SEQ ID NO:40) and LPGC (SEQ ID NO:41). In addition, impairment of the glucose tolerance test was positively correlated to insulitis, but negatively correlated to the number of functional beta cells; also this test showed that NOD mice successfully treated with NMPF were tolerant for glucose as compared to the PBS group. Our results show that PBS treated NOD mice were all diabetic at the age of 23 weeks. Whereas, NOD mice treated three times a week for two weeks with NMPF showed profound inhibition of diabetes development. The strongest anti-diabetic effects were seen with NMPF-1, -4, -5, -6, -7, -65, -66 and commercial hCG preparation (PREGNYL, batch no. 235863). These mice had a low fasting blood glucose level and were tolerant for glucose (data partially shown). However, NMPF-71 showed no effect on the incidence of diabetes, while NMPF-64 and NMPF-11 had a moderate anti-diabetic effect.

NO Experiment

NO production is a central mediator of the vascular and inflammatory response. Our results show that macrophages (RAW 264.7) stimulated with LPS produce large amounts of NO. However, these cells co-stimulated with most of the NMPF peptides (NMPF peptides 1 to 14, 43 to 66 and 69) even in a very low dose (1 pg/ml) inhibited the production of NO.

Results

apoE Experiment

The invention provides a method and a composition of the invention for the treatment of conditions that are associated with dysfunctional LDL receptors such as apoE and other members of the apolipoprotein family. In particular, use of a composition of the invention comprising GVLPALPQ (SEQ ID NO:33) (NMPF-5) and/or VLPALP (SEQ ID NO:3) (NMPF-6) or a functional analogue or derivative thereof is preferred. Groups of apoE deficient mice (n=6 per group) were fed a high cholesterol food and given PBS or NMPF every other day intraperitoneally. After 2.5 weeks, body weight was determined as shown in the Table below.

| | Average | | |
|---|---|---|---|
| | Weight (g) | SD (g) | p-value |
| ApoE-/-PBS | 31.667 | 1.007 | |
| ApoE-/-NMPF-4 | 31.256 | 1.496 | 0.536 |
| ApoE-/-NMPF-5 | 29.743 | 1.160 | 0.019 |
| Background/PBS | 26.760 | 1.582 | 10⁻⁶ |
| ApoE-/-NMPF-6 | 29.614 | 1.064 | 0.004 |

TABLE 1
Results of shock experiments in mice

(HRS)

| | | | |
|---|---|---|---|
| 0 | 16 | 40 | 72 |

| | |
|---|---|
| TEST SUBSTANCE | % SURVIVAL IN TIME |

| | | | | |
|---|---|---|---|---|
| PBS | 100 | 100 | 67 | 17 |
| PG23 | 100 | 100 | 100 | 100 |
| PG25 | 100 | 83 | 83 | 83 |

| | |
|---|---|
| PEPTIDE | |
| NMPF | SEQUENCE |

| | | | | | | |
|---|---|---|---|---|---|---|
| 1 | VLPALPQVVC | (SEQ ID NO:20) | 100 | 100 | 50 | 17 |
| 2 | LQGVLPALPQ | (SEQ ID NO:49) | 100 | 67 | 0 | 0 |
| 3 | LQG | | 100 | 83 | 20 | 17 |
| 4 | LQGV | (SEQ ID NO:1) | 100 | 100 | 100 | 100 |
| 5 | GVLPALPQ | (SEQ ID NO:33) | 100 | 100 | 80 | 17 |
| 6 | VLPALP | (SEQ ID NO:3) | 100 | 100 | 100 | 100 |
| 7 | VLPALPQ | (SEQ ID NO:168) | 100 | 83 | 0 | 0 |
| 8 | GVLPALP | (SEQ ID NO:32) | 100 | 100 | 83 | 67 |
| 9 | VVC | | 100 | 100 | 50 | 50 |
| 11 | MTRV | (SEQ ID NO:42) | 100 | 100 | 67 | 50 |
| 12 | MTR | | 100 | 100 | 67 | 50 |
| 13 | LQGVLPALPQVVC | (SEQ ID NO:34) | 100 | 100 | 100 | 100 |
| 14 | (CYCLIC) LQGVLPALPQVVC | (SEQ ID NO:34) | 100 | 83 | 83 | 83 |
| 64 | LPGCPRGVNPVVS | (SEQ ID NO:40) | 100 | 100 | 100 | 100 |
| 66 | LPGC | (SEQ ID NO:41) | 100 | 100 | 100 | 100 |

TABLE 2
Additional results of shock experiments

| NMPF SEQUENCE ID: | | |
|---|---|---|
| | | ANTI-SHOCK EFFECT |
| LQGV | (SEQ ID NO:1) | + + + |
| AQGV | (SEQ ID NO:2) | + + + |
| LQGA | (SEQ ID NO:19) | + + + |
| VLPALP | (SEQ ID NO:3) | + + + |
| ALPALP | (SEQ ID NO:21) | + + |
| VAPALP | (SEQ ID NO:22) | + + |
| ALPALPQ | (SEQ ID NO:23) | + + |
| VLPAAPQ | (SEQ ID NO:24) | + + |
| VLPALAQ | (SEQ ID NO:25) | + + + |
| | | SHOCK ACCELERATING EFFECT |
| LAGV | (SEQ ID NO:26) | + + + |
| LQAV | (SEQ ID NO:52) | + + + |
| VLAALP | (SEQ ID NO:27) | + + + |
| VLPAAP | (SEQ ID NO:117) | + + + |
| VLPALA | (SEQ ID NO:28) | + + + |
| VLPALPQ | (SEQ ID NO:29) | + + + |
| VLAALPQ | (SEQ ID NO:30) | + + + |
| VLPALPA | (SEQ ID NO:31) | + + + |

TABLE 3
Further additional results of shock experiments

| % SURVIVAL IN TIME (HRS) | |
|---|---|
| Tx | Tx |

| NMPF PEPTIDES | 0 | 14 | 24 | 48 |
|---|---|---|---|---|
| PBS | 100 | 100 | 100 | 0 |
| NMPF-3 | 100 | 100 | 100 | 0 |
| NMPF-5 | 100 | 100 | 100 | 100 |
| NMPF-7 | 100 | 100 | 100 | 67 |
| NMPF-8 | 100 | 100 | 100 | 100 |
| NMPF-9 | 100 | 100 | 100 | 100 |
| NMPF-11 | 100 | 100 | 100 | 100 |
| NMPF-12 | 100 | 100 | 100 | 100 |
| NMPF-43 | 100 | 100 | 100 | 100 |
| NMPF-45 | 100 | 100 | 100 | 100 |
| NMPF-46 | 100 | 100 | 100 | 100 |
| NMPF-50 | 100 | 100 | 100 | 100 |
| NMPF-53 | 100 | 100 | 100 | 100 |
| NMPF-58 | 100 | 100 | 100 | 100 |
| NMPF-60 | 100 | 100 | 100 | 100 |

TABLE 4
Further additional results

| | SICKNESS SCORES | | | |
|---|---|---|---|---|
| | Tx | | Tx | |
| NMPF PEPTIDES | 0 | 14 | 24 | 48 |
| PBS | 0, 0, 0, 0, 0, 0 | 5, 5, 5, 5, 4, 4 | 5, 5, 5, 5, 5, 5 | †††††† |
| NMPF-3 | 0, 0, 0, 0, 0, 0 | 3, 3, 3, 3, 3, 4 | 4, 4, 4, 4, 4, 4 | †††††† |
| NMPF-5 | 0, 0, 0, 0, 0, 0 | 5, 5, 5, 5, 5, 5 | 5, 5, 5, 5, 5, 5 | 2, 2, 2, 2, 2, 2 |
| NMPF-7 | 0, 0, 0, 0, 0, 0 | 1, 1, 4, 4, 4, 4 | 5, 5, 5, 5, 5, 5 | 2, 2, 2, 2, †† |
| NMPF-8 | 0, 0, 0, 0, 0, 0 | 3, 3, 5, 5, 5, 5 | 5, 5, 5, 5, 5, 5 | 2, 2, 4, 4, 4, 5 |
| NMPF-9 | 0, 0, 0, 0, 0, 0 | 3, 3, 4, 4, 5, 5 | 2, 2, 2, 2, 2, 2 | 1, 1, 2, 2, 2, 2 |
| NMPF-11 | 0, 0, 0, 0, 0, 0 | 1, 1, 3, 3, 4, 4, | 2, 2, 2, 2, 4, 4 | 1, 1, 1, 1, 1, 1 |
| NMPF-12 | 0, 0, 0, 0, 0, 0 | 1, 1, 1, 1, 3, 3 | 1, 1, 1, 1, 1, 1 | 1, 1, 1, 1, 1, 1 |
| NMPF-43 | 0, 0, 0, 0, 0, 0 | 1, 1, 4, 4, 4, 4 | 1, 1, 1, 1, 3, 3 | 2, 2, 2, 2, 2, 2 |
| NMPF-45 | 0, 0, 0, 0, 0, 0 | 5, 5, 5, 5, 4, 4 | 3, 3, 4, 4, 5, 5 | 2, 2, 4, 4, 5, 5 |
| NMPF-46 | 0, 0, 0, 0, 0, 0 | 1, 1, 2, 2, 3, 3 | 1, 1, 2, 2, 2, 2 | 1, 1, 1, 1, 1, 1 |
| NMPF-50 | 0, 0, 0, 0, 0, 0 | 1, 1, 1, 1, 3, 3 | 2, 2, 2, 2, 3, 3 | 1, 1, 1, 1, 1, 1 |
| NMPF-53 | 0, 0, 0, 0, 0, 0 | 5, 5, 5, 5, 5, 5 | 5, 5, 5, 5, 5, 5 | 1, 1, 2, 2, 2, 2 |
| NMPF-58 | 0, 0, 0, 0, 0, 0 | 5, 5, 5, 5, 3, 3 | 5, 5, 5, 5, 3, 3 | 1, 1, 1, 1, 1, 1 |
| NMPF-60 | 0, 0, 0, 0, 0, 0 | 1, 1, 4, 4, 2, 2 | 2, 2, 2, 2, 4, 4 | 1, 1, 1, 1, 1, 1 |

TABLE 5
Summary of results of the various peptides in the various experiments.

| | | | | | | |
|---|---|---|---|---|---|---|
| ID | SEQUENCE | SEPSIS | ANGIOGENESIS | CAO | DC | NOD |

| | | | | | | | |
|---|---|---|---|---|---|---|---|
| NMPF-1 | VLPALPQVVC | (SEQ ID NO:20) | − + | | + | + | |
| NMPF-2 | LQGVLPALPQ | (SEQ ID NO:49) | − + | | | + | |
| NMPF-3 | LQG | | − + | + | + | + | |
| NMPF-4 | LQGV | (SEQ ID NO:1) | + | + | + | + | |
| NMPF-5 | GVLPALPQ | (SEQ ID NO:33) | − + | | | + | |
| NMPF-6 | VLPALP | (SEQ ID NO:3) | + | + | + | + | |
| NMPF-7 | VLPALPQ | (SEQ ID NO:29) | + | + | | + | |
| NMPF-8 | GVLPALP | (SEQ ID NO:32) | − + | | | + | |
| NMPF-9 | VVC | | + | + | | + | |
| NMPF-10 | QVVC | (SEQ ID NO:43) | | | | | |
| NMPF-11 | MTRV | (SEQ ID NO:42) | + | + | | + | + |
| NMPF-12 | MTR | | − + | + | | + | |
| NMPF-13 | LQGVLPALPQVVC | (SEQ ID NO:34) | + | | | + | |
| NMPF-14 | cyclic-LQGVLPALPQVVC | (SEQ ID NO:34) | + | | | | |
| NMPF-43 | AQG | | + | + | | + | |
| NMPF-44 | LAG | | | + | | | |
| NMPF-45 | LQA | | + | + | | | |
| NMPF-46 | AQGV | (SEQ ID NO:2) | + | + | | + | |
| NMPF-47 | LAGV | (SEQ ID NO:26) | − + | | + | + | |
| NMPF-48 | LQAV | (SEQ ID NO:52) | | | | | |
| NMPF-49 | LQGA | (SEQ ID NO:19) | + | | | | |
| NMPF-50 | ALPALP | (SEQ ID NO:21) | + | | | + | |
| NMPF-51 | VAPALP | (SEQ ID NO:22) | + | + | | | |
| NMPF-52 | VLAALP | (SEQ ID NO:27) | | | | | |
| NMPF-53 | VLPAAP | (SEQ ID NO:117) | + | | | + | |
| NMPF-54 | VLPALA | (SEQ ID NO:28) | | | | | |
| NMPF-55 | ALPALPQ | (SEQ ID NO:23) | + | | | | |
| NMPF-56 | VAPALPQ | (SEQ ID NO:173) | | | | | |
| NMPF-57 | VLAALPQ | (SEQ ID NO:30) | | | | | |
| NMPF-58 | VLPAAPQ | (SEQ ID NO:24) | + | | | + | |
| NMPF-59 | VLPALAQ | (SEQ ID NO:25) | + | + | | | |
| NMPF-60 | VLPALPA | (SEQ ID NO:31) | + | | | + | |
| NMPF-61 | VVCNYRDVRFESIRLPGCPRGVNPVVSYAVALSCQCAL | (SEQ ID NO:35) | − + | | + | | |
| NMPF-62 | VVCNYRDVRFESIRLPGCPRGVNPVVSYAVALSCQ | (SEQ ID NO:169) | | | | | |
| NMPF-63 | SIRLPGCPRGVNPVVS | (SEQ ID NO:39) | − + | | | | |
| NMPF-64 | LPGCPRGVNPVVS | (SEQ ID NO:40) | | | + | | |
| NMPF-65 | CPRGVNPVVS | (SEQ ID NO:50) | | | | | |
| NMPF-66 | LPGC | (SEQ ID NO:41) | + | + | + | | |
| NMPF-67 | CPRGVNP | (SEQ ID NO:170) | | | | | |
| NMPF-68 | PGCP | (SEQ ID NO:10) | − + | | | | |
| NMPF-69 | RPRCRPINATLAVEKEGCPVCITVNTTICAGYCPT | (SEQ ID NO:45) | | | | | |
| NMPF-70 | MTRVLQGVLPALPQ | (SEQ ID NO:171) | − + | | | | |
| NMPF-71 | MTRVLPGVLPALPQVVC | (SEQ ID NO:174) | − + | | | | |
| NMPF-74 | CALCRRSTTDCGGPKDHPLTC | (SEQ ID NO:46) | | | | | |
| NMPF-75 | SKAPPPSLPSPSRLPGPC | (SEQ ID NO:172) | | | | | |
| NMPF-76 | TCDDPRFQDSSSSKAPPPSLPSPSRLPGPSDTPILPQ | (SEQ ID NO:48) | | | | | |

+ = effects;
− + = variable effect;
no entry is no effect or not yet tested when table was assembled

TABLE 6
MODULATION OF NO AND/OR TNF-α

| | | | | |
|---|---|---|---|---|
| ID | SEQUENCE | TNF-A | NO | TNF-A and NO |

| | | | | | |
|---|---|---|---|---|---|
| NMPF-1 | VLPALPQVVC | (SEQ ID NO:20) | + + | + + + + | + + + + |
| NMPF-2 | LQGVLPALPQ | (SEQ ID NO:49) | − + | + + + + | + + + + |
| NMPF-3 | LQG | | + | + + + + | + + + + |
| NMPF-4 | LQGV | (SEQ ID NO:1) | + + + + | + + + + | + + + + + + + |
| NMPF-5 | GVLPALPQ | (SEQ ID NO:33) | + + + + | + + + + | + + + + + + + |
| NMPF-6 | VLPALP | (SEQ ID NO:3) | + + + + | + + + + | + + + + + + + |
| NMPF-7 | VLPALPQ | (SEQ ID NO:29) | + + + + | + + + + | + + + + + + + |
| NMPF-8 | GVLPALP | (SEQ ID NO:32) | + + + + | + + + + | + + + + + + + |
| NMPF-9 | VVC | | + + + + | + + + + | + + + + + + + |
| NMPF-10 | QVVC | (SEQ ID NO:43) | + + + + | + + + | + + + + |
| NMPF-11 | MTRV | (SEQ ID NO:42) | + + + + | + + + + | + + + + |
| NMPF-12 | MTR | | + + + + | + + + + | + + + + |
| NMPF-13 | LQGVLPALPQVVC | (SEQ ID NO:34) | + + | + + + + | + + + + |
| NMPF-14 | cyclic-LQGVLPALPQVVC | (SEQ ID NO:34) | + + | + + + + | + + + + |
| NMPF-43 | AQG | | + + + + | + + + + | + + + + + + + |
| NMPF-44 | LAG | | − + | + + + + | + + + + |
| NMPF-45 | LQA | | + + + + | + + + + | + + + + + + + |
| NMPF-46 | AQGV | (SEQ ID NO:2) | + + + + | + + + + | + + + + + + + |
| NMPF-47 | LAGV | (SEQ ID NO:26) | + + | + + + + | + + + + |
| NMPF-48 | LQAV | (SEQ ID NO:52) | + + | + + + + | + + + + |
| NMPF-49 | LQGA | (SEQ ID NO:19) | + + | + + + + | + + + + |
| NMPF-50 | ALPALP | (SEQ ID NO:21) | + + + + | + + + + | + + + + + + + |
| NMPF-51 | VAPALP | (SEQ ID NO:22) | + | + + + | + + + + |
| NMPF-52 | VLAALP | (SEQ ID NO:27) | + + | + + + + | + + + + |
| NMPF-53 | VLPAAP | (SEQ ID NO:117) | + + + + | + + + + | + + + + + + + |
| NMPF-54 | VLPALA | (SEQ ID NO:28) | + | + + + + | + + + + |
| NMPF-55 | ALPALPQ | (SEQ ID NO:23) | + | + + + + | + + + + |
| NMPF-56 | VAPALPQ | (SEQ ID NO:173) | − + | + + + + | + + + + |
| NMPF-57 | VLAALPQ | (SEQ ID NO:30) | + | + + + + | + + + + |
| NMPF-58 | VLPAAPQ | (SEQ ID NO:24) | + + + + | + + + + | + + + + + + + |
| NMPF-59 | VLPALAQ | (SEQ ID NO:25) | + + | + + + + | + + + + |
| NMPF-60 | VLPALPA | (SEQ ID NO:31) | + + + + | + + + + | + + + + + + + |
| NMPF-61 | VVCNYRDVRFESIRLPGCPRGVNPVVSYAVA | (SEQ ID NO:35) | − + | + + + + | + + + + |
| | LSCQCAL | | | | |
| NMPF-62 | VVCNYRDVRFESIRLPGCPRGVNPVVSYAVA | (SEQ ID NO:169) | − + | + + + | + + + + |
| | LSCQ | | | | |
| NMPF-63 | SIRLPGCPRGVNPVVS | (SEQ ID NO:39) | − + | + + | + + |
| NMPF-64 | LPGCPRGVNPVVS | (SEQ ID NO:40) | + + | + + + + | + + + + |
| NMPF-65 | CPRGVNPVVS | (SEQ ID NO:50) | + + | + + + | + + + |
| NMPF-66 | LPGC | (SEQ ID NO:41) | + + + | + + | + + + |
| NMPF-67 | CPRGVNP | (SEQ ID NO:170) | − + | + | + |
| NMPF-68 | PGCP | (SEQ ID NO:10) | + | + | + + + |
| NMPF-69 | RPRCRPINATLAVEKEGCPVCITVNTTICAGY | (SEQ ID NO:45) | − + | + + | + + |
| | CPT | | | | |
| NMPF-70 | MTRVLQGVLPALPQ | (SEQ ID NO:171) | − + | + | + |
| NMPF-71 | MTRVLPGVLPALPQVVC | (SEQ ID NO:174) | − + | − + | − + |
| NMPF-74 | CALCRRSTTDCGGPKDHPLTC | (SEQ ID NO:46) | − + | + + | + |
| NMPF-75 | SKAPPPSLPSPSRLPGPS | (SEQ ID NO:172) | + | + + | + + |
| NMPF-76 | TCDDPRFQDSSSSKAPPPSLPSPSRLPGPSDTP | (SEQ ID NO:48) | + | + | + |
| | ILPQ | | | | |
| NMPF-78 | CRRSTTDCGGPKDHPLTC | (SEQ ID NO:47) | + | + | + |

from − + to + + + + + + + indicates from barely active to very active in modulating

### Example V

TNF-αexperiment

Cell culture. The RAW 264.7 murine macrophage cell line, obtained from American Type Culture Collection (Manassas, Va., USA), is cultured at 37° C. in 5% CO₂ using DMEM containing 10% fetal calf serum (FCS), 50 U/ml penicillin, 50 μg/ml streptomycin, 0.2 M Na-pyruvate, 2 mM glutamine and 50 μM 2-mercaptoethanol (Bio Whittaker, Europe). The medium is changed every 2 days.

Cell transfection. RAW 264.7 cells are transfected with a NF-κB dependent reporter construct.

NF-κB measurements. Output from the NF-κB dependent reporter is measured. The cells (7.5×10⁵/ml) are cultured in 96-well plates in 500 μl of culture medium. The cells are stimulated with LPS (10 μg/ml) and/or a NMPF derivative (e.g., 1 μg/ml, 1 ng/ml, 1 μg/ml) for 24 hours, then the output from the NF-κB reporter is measured as appropriate for the reporter. Alternatively, NF-κB mRNA levels may be quantitated, for example by Northern blot analysis or other mRNA quantitation methods known in the art.

Peptide derivatives. Peptides derived from VLPALP (SEQ ID NO:3) are synthesized using replacement net ananlysis. A total of 120 peptides are synthesized and placed into the wells of two 96-well plates. RAW 264.7 cells in fresh media are added to the 96 well plates and the cells are stimulated with LPS (10 μg/ml) and the level of NF-κB mRNA determined at an appropriate time.

Likewise, other pro inflammatory cytokine levels may be assayed.

In another embodiment, the results are imput into a database. The database is then sorted

### Example VI

Monkey Experiment

Efficacy of NMPF, here a mixture 1:1:1 of LQGV (SEQ ID NO:1), AQGV (SEQ ID NO:2) and VLPALP (SEQ ID NO:3), administered in a gram-negative induced rhesus monkey sepsis model for prevention of septic shock.

Overwhelming inflammatory and immune responses are essential features of septic shock and play a central part in the pathogenesis of tissue damage, multiple organ failure, and death induced by sepsis. Cytokines, especially tumor necrosis factor (TNF)-α interleukin (IL)-1β, and macrophage migration inhibitory factor (MIF), have been shown to be critical mediators of septic shock. Yet, traditional anti-TNF and anti-IL-1 therapies have not demonstrated much benefit for patients with severe sepsis. We have designed peptides that block completely LPS induced septic shock in mice, even when treatment with these peptides is started up to 24 hours after LPS injection. These peptides are also able to inhibit the production of MIF. This finding provides the possibility of therapeutic use of these peptides for the treatment of patients suffering from septic shock. Since primates are evolutionary more closer to humans, we tested these peptides for their safety and effectiveness in a primate system.

EXPERIMENTAL DESIGN

| | EXPERIMENTAL | | |
|---|---|---|---|
| | TREATMENT | | |
| | (independent variable, | | |
| | e.g., placebo treated | | |
| GROUP | control group) | BIOTECHNIQUES | NUMBER |
| animal I | i.v. infusion of a lethal | Live E. coli infusion | N = 1 |
| | dose of live | Blood sampling | |
| | Escherichia. coli (10E10 | No recovery (section) | |
| | CFU/kg) + antibiotics + | | |
| | placebo treated | | |
| animal II | i.v. infusion of a lethal | Live E. coli infusion | N = 1 |
| | dose of live | Blood sampling | |
| | Escherichia. coli (10E10 | No recovery (section) | |
| | CFU/kg) + antibiotics + | | |
| | oligopeptide (5 mg/kg | | |
| | of each of 3 peptides) | | |

Only naive monkeys were used in this preclinical study to exclude any interaction with previous treatments. The animals were sedated with ketamine hydrochloride. Animals were intubated orally and allowed to breathe freely. The animals were kept anesthetized with O₂/N₂O/isoflurane. The animals received atropin as pre-medication for O₂/N₂O/isoflurane anesthesia. A level of surgical anesthesia was maintained during the 2 h infusion of E. coli and for 6 h following E. coli challenge, after which the endothracheal tubes were removed and the animals were euthanized. Before bacteria were induced, a 1 hour pre-infusion monitoring of heart-rate and blood pressure was performed.

Two rhesus monkeys were infused with a 10¹⁰ CFU per kg of the Gram negative bacterium E. coli to induce a fatal septic shock. One monkey received placebo-treatment and was sacrificed within 7 hours after infusion of the bacteria without recovery from the anesthesia. The second monkey received treatment with test compound and was sacrificed at the same time point.

In a limited dose-titration experiment performed with the same bacterium strain in 1991, the used dose proved to induce fatal shock within 8 hours. In recent experiments, a 3-fold lower dose was used inducing clear clinical and pathomorphological signs of septic shock without fatal outcome.

The monkeys were kept anesthetized throughout the observation period and sacrificed 7 hours after the start of the bacterium infusion for pathological examination. The animals underwent a gross necropsy in which the abdominal and thorax cavities were opened and internal organs examined in situ.

Full Description of the Experiment with Three Rhesus Monkeys

The study was conducted in rhesus monkeys (Maccaca mulatta). Only experimentally naive monkeys were used in the study to exclude any interaction with previous treatments. Prior to the experiment, the state of health of the animals was assessed physically by a veterinarian. All animals had been declared to be in good health and were free of pathogenic ecto- and endoparasites and common bacteriological infections: Yersinia pestis, Yersinia enterocolitica, Yersinia pseudotuberculosis, Shigella, Aeromonas hydrophilia, pathogenic Campylobacter species and Salmonella.

Reagents. The Escherichia coli strain was purchased from ATCC (E. coli; 086a: K61 serotype, ATCC 33985). In a control experiment, the strain proved equally susceptible to bactericidal factors in human and rhesus monkey serum. Prior to the experiment, a fresh culture was set-up; the E. coli strain was cultured for one day, harvested and washed thoroughly to remove free endotoxine. Prior to infusion into the animal, the number and viability of the bacteria were assessed. Serial dilutions of the E. coli stock were plated on BHI agar and cultured overnight at 37° C. The colonies on each plate were counted and the number of colony-forming units per ml was calculated. The body weight measurement of the day of the experiment was used to calculate the E. coli dose and E. coli stock was suspended in isotonic saline (N.P.B.I., Emmer-Compascuum, NL) at the concentration needed for infusion (total dose volume for infusion approximately 10 ml/kg. The E. coli suspension was kept on ice until infusion.

Antibiotic was used to synchronize the shock induction in the monkeys. Baytril (Baytril 2.5%, Bayer, Del.) was used instead of gentamycin, as the strain proved only marginally susceptible to the latter antibiotic. Individual animals were identified by a number or letter combination tattooed on the chest.

Experimental design.

| | EXPERIMENTAL | | | |
|---|---|---|---|---|
| GROUP | TREATMENT | | | |
| (number/ | (independent variable, | | | |
| letter or other | e.g., placebo treated | | | |
| identification | control group) | | NUMBER | SEX |
| Animal I | i.v. infusion of a lethal | Live E. coli infusion | N = 1 | F |
| | dose of live | Blood sampling | | |
| | Escherichia. coli (10E10 | No recovery | | |
| | CFU/kg) + antibiotic + | | | |
| | placebo treated | | | |
| Animal II | i.v. infusion of a lethal | Live E. coli infusion | N = 1 | F |
| | dose of live | Blood sampling | | |
| | Escherichia. coli (10E10 | No recovery (section) | | |
| | CFU/kg) + antibiotic + | | | |
| | NMPF-4, -6, -46; each 5 mg/kg | | | |
| Animal III | i.v. infusion of a lethal | Live E. coli infusion | N = 1 | F |
| | dose of live | Blood sampling | | |
| | Escherichia. coli (10E10 | Recovery and survival | | |
| | CFU/kg) + antibiotic + | | | |
| | NMPF-4, -6, -46; each 5 mg/kg | | | |

Anesthesia. All animals were fasted overnight prior to the experiment. On the morning of the experiment, the animals were sedated with ketamine hydrochloride (Tesink, NL) and transported to the surgery. The animal was placed on its side on a temperature-controlled heating pad to support body temperature. Rectal temperature was monitored using a Vet-OX 5700. The animals were intubated orally and were allowed to breathe freely. The animals were kept anesthesized using O₂/N₂O/isoflurane inhalation anesthesia during the E. coli infusion and the 7 hour observation period following E. coli challenge, after which the endothracheal tubes were removed and the animals were euthanized or allowed to recover from anesthesia. The femoral or the cephalic vein was cannulated and used for infusing isotonic saline, live E. coli and antibiotic administration. Insensible fluid loss was compensated for by infusing isotonic saline containing 2.5% glucose (Fresenius, 's Hertogenbosch, NL) at a rate of 3.3 ml/kg/hr.

Preparative actions. During anesthesia the animals were instrumented for measurement of blood pressure (with an automatic cuff), heart rate and body temperature. Isotonic saline was infused at 3.3 ml/kg/hr to compensate for fluid loss. Femoral vessels were cannulated for infusion of E. coli and antibiotics. Temperature-controlled heating pads were used to support body temperature. The monkeys were continuously monitored during the E. coli challenge and for the 6 hr period following E. coli administration. After 7 hrs, 2 animals (the control animal and one treated with NMPF) were sacrificed to compare the direct effect of the compound at the level of histology. The 3^{rd} animal, treated with NMPF, was allowed to recover from anesthesia and was intensively observed during the first 12 hours after recovery followed by frequent daily observation. The decision to allow the 3rd animal to recover was made after consulting with the veterinarian.

Induction of septic shock. Before the infusion of E. coli, a 1 hr pre-infusion monitoring of heart-rate and blood pressure was performed. All three animals received an i.v. injection of E. coli 086 (k61 serotype; ATCC 33985) at a lethal dose of 10×109 CFU/kg body weight. In a dose titration study with this batch performed in 1991, this bacterial dose induced lethal shock within 8 hrs after the start of the infusion. The infusion period was 2 hrs.

Antibiotics. Baytril was administered intravenously immediately after completion of the 2 h.E. coli infusion (i.v.; dose 9 mg/kg).

Treatment with NMPF. 30 minutes post-onset of E. coli infusion, the animals were administered a single intravenous bolus injection of a mixer of NMPF peptides. The peptide mixture contained the following NMPF peptides: LQGV (SEQ ID NO:1) (5 mg/kg), AQGV (SEQ ID NO:2) (5 mg/kg) and VLPALP (SEQ ID NO:3) (5 mg/kg). These NMPF peptides were dissolved in 0.9% sodium chloride for injection (N.P.B.I., Emmer Compascuum, NL).

RESULTS

Preliminary Monkey Results

An anti-shock effect of the test compound on sepsis in the monkey treated with the oligopeptide mixture, namely the inhibition of the effect of the sepsis in this early 7-hour trajectory of this primate model, was observed. Immunomodulatory effects with these peptides have been observed in vitro/ex vivo such as in T-cell assays, the inhibition of pathological Th1 immune responses, suppression of inflammatory cytokines (MIF), increase in production of anti-inflammatory cytokines (IL-10, TGF-beta) and immunomodulatory effects on antigen-presenting cells (APC) like dendritic cells and macrophages.

The following organs were weighed and a bacterial count was performed: kidneys, liver, lungs, lymph nodes, and gross lesions.

Tissues of all organs were preserved in neutral aqueous phosphate buffered 4% solution of formaldehyde. Lymphoid organs were cryopreserved. All tissues will be processed for histopathological examination.

Further Results Obtained in the Three-Monkey Experiment

Monkey 429(control). Female monkey (5.66 kg) received an i.v. injection of E. coli 086 (10E10 CFU/kg). In a dose titration study with this batch performed in 1991, this bacterial dose induced lethal shock within 8 hrs after the start of the infusion. The infusion period was 2 hrs. Baytril was administered intravenously immediately after completion of the 2 h. E. coli infusion (i.v.; dose 9 mg/kg). After the E. coli injection, the monkey was observed by the authorized veterinarian without knowing which of the monkeys received NMPF treatment. The clinical observations were as follows: vomiting, undetectable pulse, heart arythmia, abnormalities in ECG: signs of ventricle dilatation/heart decompensation (prolonged QRS complex, extra systoles), decreased blood clotting and forced respiration. In addition, there was big fluctuation in heart rate (30-150 beats per minute), collapse of both systolic and diastolic blood pressure (35/20 mmHg) and decrease in blood oxygen concentration (80-70%). Seven hours after the start of the E. coli infusion, monkey began to vomit blood and feces, and have convulsions. After final examination, the veterinarian did not give permission to let this monkey awake. At this time point, the control monkey was euthanized. Hereafter, post-mortem examination was conducted and internal organs were examined in situ. A number of internal bleedings were found by the pathologist.

Monkey 459(NMPF). Female monkey (5.44 kg) received an i.v. injection of E. coli 086 (10E10 CFU/kg). In a dose titration study with this batch performed in 1991, this bacterial dose induced lethal shock within 8 hrs after the start of the infusion. The infusion period was 2 hrs. Thirty minutes after the initiation of E. coli infusion, NMPF was i.v. injected in a single bolus injection. Baytril was administered intravenously immediately after completion of the 2 h. E. coli infusion (i.v.; dose 9 mg/kg). After the E. coli injection, this monkey was also observed by the authorized veterinarian without knowing which of the monkeys received NMPF treatment. The clinical observations were as follows: normal pulse, heart sounds normal, normal ECG, higher heart-rate but otherwise stable (180 beats per minute), no hypotension (75/30 mmHg), normal blood oxygen concentration (95-85%), lungs sound normal, normal turgor. Seven hours after the start of the E. coli infusion, the clinical condition of the monkey was stable. After final examination, the veterinarian did give permission to let this monkey awake due to her stable condition. In order to compare the hematological and immunological parameters between the control and NMPF-treated monkey, at this time point the NMPF-treated monkey 459 was euthanized. Hereafter, post-mortem examination was conducted and internal organs were examined in situ. No macroscopic internal bleedings were found by the pathologist.

Monkey 427(NMPF). Female monkey (4.84 kg) received an i.v. injection of E. coli 086 (10E10 CFU/kg). In a dose titration study with this batch performed in 1991, this bacterial dose induced lethal shock within 8 hrs after the start of the infusion. The infusion period was 2 hrs. Thirty minutes after the initiation of E. coli infusion, NMPF was i.v. injected. Baytril was administered intravenously immediately after completion of the 2 h. E. coli infusion (i.v.; dose 9 mg/kg). After the E. coli injection, this monkey was also observed by the authorized veterinarian doctor without knowing which of the monkeys received NMPF treatment. The clinical observations were as follows: normal pulse, heart sounds normal, normal ECG, moderately higher heart-rate but otherwise stable (160 beats per minute), no hypotension (70/30 mmHg), normal blood oxygen concentration (95-90%), lungs sound normal, normal turgor. Seven hours after the start of the E. coli infusion, the clinical condition of the monkey was stable. After final examination, the veterinarian did give permission to let this monkey wake up due to her stable condition. The monkey woke up quickly, she was alert and there was a slow disappearance of oedema.

### Example VII

Cells, for example, 3T3 cells, are transfected with an expression cassette that expresses the peptide of SEQ ID NO:1 or SEQ ID NO:34, which is found to increase production of NF-κB. Production of NF-κB may be assayed by electrophoretic mobility shift assays. For example, an oligonucleotide representing NF-κB binding sequence such as (5′-AGC TCA GAG GGG GAC TTT CCG AGA G-3′) (SEQ ID NO: 51) are synthesized. Hundred pico mol sense and antisense oligo are annealed and labeled with α-³²P-dATP using T4 polynucleotide kinase according to manufacture's instructions (Promega, Madison, Wis.). Cytosolic extract or nuclear extract (5-7.5 μg) from cells expressing the regulatory peptide or from untreated cells (e.g., as a control) is incubated for 30 minutes with 75000 cpm probe in binding reaction mixture (20 μl) containing 0.5 μg poly dI-dC (Amersham Pharmacia Biotech) and binding buffer BSB (25 mM MgC12, 5 mM CaCl₂, 5 mM DTT and 20% Ficoll) at room temperature. Or cytosolic and nuclear extract from untreated cells or from cells treated with stimuli could also be incubated with probe in binding reaction mixture and binding buffer. The DNA-protein complexes are resolved from free oligonucleotide by electrophoresis in a 4-6% polyacrylamide gel (150 V, 2-4 hours). The gel is then dried and exposed to x-ray film.

Increased Expression

Candidate Peptides can also be biotinylated and incubated with cells. Cells are then washed with phosphate-buffered saline, harvested in the absence or presence of certain stimulus (LPS, PHA, TPA, anti-CD3, VEGF, TSST-1, VIP or know drugs etc.). After culturing, cells are lysed and cells lysates (whole lysate, cytosolic fraction or nuclear fraction) containing 200 micro gram of protein are incubated with 50 miroliters of Neutr-Avidin-plus beads for 1 h at 4° C. with constant shaking. Beads are washed five times with lysis buffer by centrifugation at 6000 rpm for 1 min. Proteins are eluted by incubating the beads in 0.05 N NaOH for 1 min at room temperature to hydrolyze the protein-peptide linkage and analyzed by SDS-polyacrylamide gel electrophoresis followed by immunoprecipitated with agarose-conjugated anti-NF-κB subunits antibody or immunoprecipitated with antibody against the target, where that target is other than NF-κB. After hydrolyzing the protein-peptide linkage, the sample may be analyzed on HPLS and mass-spectrometry. Purified NF-κB subunits or cell lysate interaction with biotinylated regulatory peptide can be analyzed using biosensor technology. Peptides can be labeled with FITC and incubated with cells in the absence or presence of different stimulus. After culturing, cells can be analyzed with fluorescent microscopy, confocal microscopy, flow cytometry (cell membrane staining and/or intracellular staining) or cells lysates are made and analyzed on HPLC and mass-spectrometry. NF-κB transfected (reporter gene assay) cells and gene array technology can be used to determine the regulatory effects of peptides.

An exemplary embodiment of the invention provides an improvement in a method of screening a candidate compound for biological activity by screening the compound in a cell line, the improvement comprising: screening said candidate compound in a cell line wherein said cell line has been contacted with at least one exogenously added biologically active peptide having gene regulatory activity.

### Example VIII

RNA Extraction and DNA Microarray Procedures

RNA was isolated using RNeasy columns as described by the manufacturer (Qiagen, Hilden, Germany). The integrity of the RNA was tested on 1% formaldehyde containing agarose gels. A total of 5 μg of RNA was used to generate ds cDNA using superscript reverse transcriptase and a T7-oligodT primer. The resulting cDNA was used in an in vitro cRNA reaction using T7 RNA polymerase and biotinylated ribonucleotides employing an ENZO kit (ENZO, Farmingdale, N.Y., USA). The biotinylated cRNA was cleaned-up using RNeasy spin columns (Qiagen) and quantified by spectrophotometric methods. An adjusted cRNA yield was calculated to reflect carryover of unlabeled total RNA. Fragmentation of 20 μg cRNA was performed at 95° C. for 35 min. Fragmented cRNA (10 μg) was subsequently hybridized for 16 h to U95A microarrays (Affymetrix) at 45° C. After washing and staining with PE-conjugated streptavidin, the arrays were scanned in an HPAffymetrix scanner at 570 nm using a kryptonargon laser.

The scanned images were analyzed using Affymetrix Microarray suite 4.2 software, using either LPS, PHA or only PBS treated sample as baseline.

Ratios between the 5′ oligonucleotides and the 3′ oligonucleotides of GAPDH transcripts were <1.5 (usually 0.91.1.), indicating that the amount of labeling was equally distributed over the RNA molecules. This implies that no major degradation of RNA occurred. In comparison experiments, care was taken that the scaling factor, noise, and presence calls were all comparable.

Analysis of the result was performed as follows: for example two arrays were compared, LPS and PBS treated sample, using Affymetrix Microarray suite 4.2. PBS array was used as baseline. Genes absent in both arrays and genes not changed were deleted. The ten most decreased and increased genes were collected and are described in tables. Affymetrix probeset descriptions are collected from either the internet site of Affymetrix (affymetrix.com/index.affx) or from EASE version 2.0 software. A description of the gene accession numbers are also shown at nucleotide database of internet site (ncbi.nlm.nih.gov/entrez).

The peptides that are used in the present experimental design are usually of synthetic origin and produced by chemical synthesis. Nevertheless, where desirable and appropriate, the peptides may also be isolated by fractionating a biological sample or produced by recombinant techniques known in the art. In addition, the peptides, whether chemically synthesized or produced by recombinant techniques, may contain modifications known in the art.

Based on the disclosure herein, a person of ordinary skill in the art will recognize that the peptides tested, may be of any length, particularly desirable are trimers and tetramers, but any peptide fragment may be tested using the methodology disclosed herein. Furthermore, the results of the test may beneficially be compiled into a database, which may then be searched and/or used to identify peptides with desirable activities.

Results:

| | | |
|---|---|---|
| | Times | |
| | change | |
| Affymetrix | (− = decreased) | |
| Probesets | (+ = increased) | Discription |

PBMC + PHA/IL2 + VVC vs PBMC + PHA/IL2 + PBS

| | | | |
|---|---|---|---|
| 37627_g_at | −9.4 | Cluster Incl. D78261: Human ICSAT | |
| | | transcription factor mRNA, partial cds, | |
| | | similar to mouse Pip | |
| 31491_s_at | −8.8 | Cluster Incl. X98175: H. sapiens mRNA | |
| | | for MACH-beta-2 protein | |
| 1571_f_at | −8 | L49229 | DEFINITION = HUMRB1AAD |
| | | with a 3 bp deletion in exon 22 (L11910 | L Homo sapiens |
| | | bases 161855-162161) | retinoblastoma susceptibility |
| | | | protein (RB1) gene, |
| 1219_at | −7.2 | X15949 | DEFINITION = HSIRF2 |
| | | | Human mRNA for interferon |
| | | | regulatory factor-2 (IRF-2) |
| 39948_at | −5.8 | Cluster Incl. AI693307: wd91b01.x1 | |
| | | Homo sapiens cDNA, 3 end | |
| 31534_at | −5.8 | Cluster Incl. M30607: Human zinc finger | |
| | | protein Y-linked (ZFY) mRNA, complete | |
| | | cds | |
| 35440_g_at | −5.6 | Cluster Incl. D26121: Human mRNA for | |
| | | ZFM1 protein alternatively spliced | |
| | | product, complete cds | |
| 303_at | −5.6 | Guanine Nucleotide Exchange Factor 1 | |
| 33152_at | −5.6 | Cluster Incl. AF047432: Homo sapiens | |
| | | ADP-ribosylation factor mRNA, | |
| | | complete cds | |
| 37626_at | −5.4 | Cluster Incl. D78261: Human ICSAT | |
| | | transcription factor mRNA, partial cds, | |
| | | similar to mouse Pip | |
| 31811_r_at | 9.6 | Cluster Incl. L11667: Human cyclophilin- | |
| | | 40 mRNA, complete cds | |
| 33526_at | 7.2 | Cluster Incl. U50146: Human type 2 | |
| | | neuropeptide Y receptor (NPY Y2) | |
| | | gene, partial | |
| 35950_at | 6.6 | Cluster Incl. U90841: Homo sapiens | |
| | | SSX4 (SSX4) mRNA, complete cds | |
| 32555_at | 6.6 | Cluster Incl. AA149637: zl39b08.s1 | |
| | | Homo sapiens cDNA, 3 end | |
| 34610_at | 6.4 | Cluster Incl. W25845: 13h9 Homo | |
| | | sapiens cDNA | |
| 31791_at | 6.2 | Cluster Incl. Y16961: Homo sapiens | |
| | | mRNA for KET protein | |
| 40627_at | 6 | Cluster Incl. AI192108: qa06d10.x1 | |
| | | Homo sapiens cDNA, 3 end | |
| 40858_at | 5.4 | Cluster Incl. M34715: Human pregnancy- | |
| | | specific beta-1-glycoprotein mRNA | |
| | | PSG95, complete cds | |
| 32247_at | 4.6 | Cluster Incl. X53795: Human R2 mRNA | |
| | | for an inducible membrane protein | |
| 1894_f_at | 4.6 | Neurofibromatosis 2 Tumor Suppressor | |

PBMC + LPS + MTR vs PBMC + LPS + PBS

| | | | |
|---|---|---|---|
| 34887_at | −9.6 | Cluster Incl. N92548: zb29g04.s1 Homo | clone = IMAGE-305046 |
| | | sapiens cDNA, 3 end | |
| 544_at | −9.2 | DEFINITION = S76638 p50-NF-kappa B | |
| | | homolog [human, peripheral blood T | |
| | | cells, mRNA | |
| 32737_at | −8.6 | Cluster Incl. M64595: Human small G | |
| | | protein (Gx) mRNA, 3 end | |
| 2049_s_at | −8 | DEFINITION = HUMJUNCAA Human | |
| | | transactivator (jun-B) gene, complete | |
| | | cds | |
| 146_at | −7.8 | DEFINITION = HSU81802 Human Ptdlns | |
| | | 4-kinase (PI4Kb) mRNA, complete cds | |
| 32844_at | −7.6 | Cluster Incl. AF104913: Homo sapiens | |
| | | eukaryotic protein synthesis initiation | |
| | | factor mRNA, complete cds | |
| 499_at | −7.6 | DEFINITION = HSU33822 Human tax1- | |
| | | binding protein TXBP181 mRNA, | |
| | | complete cds | |
| 41198_at | −7.6 | Cluster Incl. AF055008: Homo sapiens | |
| | | clone 24720 epithelin 1 and 2 mRNA, | |
| | | complete cds | |
| 32640_at | −7.2 | Cluster Incl. M24283: Human major | |
| | | group rhinovirus receptor (HRV) mRNA, | |
| | | complete cds | |
| 1635_at | −7.2 | U07563 | DEFINITION = HSABLGR3 |
| | | | Human proto-oncogene |
| | | | tyrosine-protein kinase (ABL) |
| | | | gene |
| 38302_at | 8.4 | Cluster Incl. AF027219: Homo sapiens | |
| | | ZNF202 beta (ZNF202) mRNA, | |
| | | complete cds | |
| 40387_at | 6.8 | Cluster Incl. U80811: Human | |
| | | lysophosphatidic acid receptor homolog | |
| | | mRNA, complete cds | |
| 31961_r_at | 6.4 | Cluster Incl. AF070579: Homo sapiens | |
| | | clone 24487 mRNA sequence | |
| 1623_s_at | 6.4 | Tyrosine Kinase Fer | |
| 34577_at | 6 | Cluster Incl. U10694: Human MAGE-9 | |
| | | antigen (MAGE9) gene, complete cds | |
| 35003_at | 5.6 | Cluster Incl. AA534868: nf82b01.s1 | |
| | | Homo sapiens cDNA, 3 end | |
| 38627_at | 5.6 | Cluster Incl. M95585: Human hepatic | |
| | | leukemia factor (HLF) mRNA, complete | |
| | | cds | |
| 31923_f_at | 5.4 | Cluster Incl. U60269: Human | |
| | | endogenous retrovirus HERV-K(HML6) | |
| | | proviral clone HML6.17 putative | |
| | | polymerase and envelope genes | |
| 37615_at | 3.6 | Cluster Incl. D86962: Human mRNA for | |
| | | KIAA0207 gene, complete cds | |
| 36178_at | 3.6 | Cluster Incl. U23143: Human | |
| | | mitochondrial serine | |
| | | hydroxymethyltransferase gene, nuclear | |
| | | encoded mitochondrion protein | |

PBMC + LPS vs PBMC + PBS

| | | |
|---|---|---|
| 41814_at | −5.2 | Cluster Incl. M29877: Human alpha-L- |
| | | fucosidase, complete cds |
| 859_at | −3.8 | DEFINITION = HSU03688 Human dioxin- |
| | | inducible cytochrome P450 (CYP1B1) |
| | | mRNA, complete cds |
| 1894_f_at | −3 | Neurofibromatosis 2 Tumor Suppressor |
| 34498_at | −2.8 | Cluster Incl. D89974: Homo sapiens |
| | | mRNA for glycosylphosphatidyl inositol- |
| | | anchored protein GPI-80, complete cds |
| 40071_at | −2.6 | Cluster Incl. U03688: Human dioxin- |
| | | inducible cytochrome P450 (CYP1B1) |
| | | mRNA, complete cds |
| 39036_g_at | −2.6 | Cluster Incl. AF006010: Human |
| | | progestin induced protein (DD5) mRNA, |
| | | complete cds |
| 34891_at | −1.8 | Cluster Incl. |
| | | AI540958: PEC1.2_15_H01.r Homo |
| | | sapiens cDNA, 5 end |
| 39728_at | −1.8 | Cluster Incl. J03909: Human gamma- |
| | | interferon-inducible protein (IP-30) |
| | | mRNA, complete cds |
| 38661_at | 10 | Cluster Incl. X75315: H. sapiens seb4B |
| | | mRNA |
| 1306_at | 9.8 | DEFINITION = HUMEIF4G Human |
| | | mRNA for eukaryotic initiation factor 4 |
| | | gamma (eIF-4 gamma) |
| 40362_at | 9 | Cluster Incl. X61498: H. sapiens mRNA |
| | | for NF-kB subunit |
| 563_at | 9 | DEFINITION = HUMNKSFP40 Human |
| | | natural killer cell stimulatory factor |
| | | (NKSF) mRNA, complete cds, clone p40 |
| 1069_at | 8.6 | DEFINITION = HSU04636 Human |
| | | cyclooxygenase-2 (hCox-2) gene, |
| | | complete cds |
| 32640_at | 8.4 | Cluster Incl. M24283: Human major |
| | | group rhinovirus receptor (HRV) mRNA, |
| | | complete cds |
| 446_at | 8.4 | DEFINITION = HSU89896 Homo sapiens |
| | | casein kinase I gamma 2 mRNA, |
| | | complete cds |
| 544_at | 8.4 | DEFINITION = S76638 p50-NF-kappa B |
| | | homolog [human, peripheral blood T |
| | | cells, mRNA, 3113 nt] |
| 2049_s_at | 8.4 | DEFINITION = HUMJUNCAA Human |
| | | transactivator (jun-B) gene, complete |
| | | cds |
| 38299_at | 8.4 | Cluster Incl. X04430: Human IFN-beta |
| | | 2a mRNA for interferon-beta-2 |
| 36178_at | 3.6 | Cluster Incl. U23143: Human |
| | | mitochondrial serine |
| | | hydroxymethyltransferase gene, nuclear |
| | | encoded mitochondrion protein |

PBMC + LPS + MTRV vs PBMC + LPS + PBS

| | | | |
|---|---|---|---|
| 41672_at | −3.4 | Cluster Incl. AF007128: Homo sapiens | |
| | | clone 23870 mRNA sequence | |
| 37635_at | −2.6 | Cluster Incl. L09190: Human trichohyalin | |
| | | (TRHY) gene, complete cds | |
| 39797_at | −2.4 | Cluster Incl. AB002347: Human mRNA | |
| | | for KIAA0349 gene, partial cds | |
| 36538_at | −2.1 | Cluster Incl. AB018314: Homo sapiens | |
| | | mRNA for KIAA0771 protein, partial cds | |
| 35668_at | −1.9 | Cluster Incl. AJ001014: Homo sapiens | |
| | | mRNA encoding RAMP1 | |
| 37751_at | −1.6 | Cluster Incl. D87444: Human mRNA for | |
| | | KIAA0255 gene, complete cds | |
| 39835_at | −1.4 | Cluster Incl. U93181: Homo sapiens | |
| | | nuclear dual-specificity phosphatase | |
| | | (SBF1) mRNA, partial cds | |
| 38549_at | −1.3 | Cluster Incl. AF026941: Homo sapiens | |
| | | cig5 mRNA, partial sequence | |
| 37748_at | −1.2 | Cluster Incl. D86985: Human mRNA for | |
| | | KIAA0232 gene, complete cds | |
| 36808_at | −1.1 | Cluster Incl. AF001846: Homo sapiens | |
| | | lymphoid phosphatase LyP1 mRNA, | |
| | | complete cds | |
| 38484_at | 4.5 | Cluster Incl. D21267: Homo sapiens | |
| | | mRNA, complete cds | |
| 36788_at | 4.3 | Cluster Incl. U66033: Human glypican-5 | |
| | | (GPC5) mRNA, complete cds | |
| 35435_s_at | 3.7 | Cluster Incl. AF001903: Human 3- | |
| | | hydroxyacyl-CoA dehydrogenase, | |
| | | isoform 2 mRNA, complete cds | |
| 41493_at | 3.3 | Cluster Incl. AI094610: oy64f07.s1 Homo | |
| | | sapiens cDNA, 3 end | |
| 39308_r_at | 3 | Cluster Incl. X81637: H. sapiens clathrin | |
| | | light chain b gene | |
| 1481_at | 2.8 | L23808 | DEFINITION = HUMHME |
| | | | Human metalloproteinase |
| | | | (HME) mRNA, complete cds |
| 798_at | 2.1 | X74330 | DEFINITION = HSPRIM1 |
| | | | H. sapiens mRNA for DNA |
| | | | primase (subunit p48) |
| 39315_at | 2 | Cluster Incl. D13628: Human mRNA for | |
| | | KIAA0003 gene, complete cds | |
| 32791_at | 1.7 | Cluster Incl. L19183: Human MAC30 | |
| | | mRNA, 3 end | |
| 33559_at | 1.6 | Cluster Incl. U61412: Human non- | |
| | | receptor type protein tyrosine kinase | |
| | | (PTK6) gene | |

PBMC + LPS + MTRVLQGVLPALPQVVC vs PBMC + LPS + PBS

| | | |
|---|---|---|
| 37215_at | −7.8 | Cluster Incl. AF046798: untitled |
| 37050_r_at | −7.6 | Cluster Incl. AI130910: qb81g08.x1 |
| | | Homo sapiens cDNA, 3 end |
| 35232_f_at | −7.2 | Cluster Incl. AI056696: oz26h05.x1 |
| | | Homo sapiens cDNA, 3 end |
| 40556_at | −7 | Cluster Incl. D42073: Human mRNA for |
| | | reticulocalbin, complete cds |
| 40438_at | −6.8 | Cluster Incl. D87930: Homo sapiens |
| | | mRNA for myosin phosphatase target |
| | | subunit 1 (MYPT1) |
| 40133_s_at | −6.8 | Cluster Incl. W28944: 54h12 Homo |
| | | sapiens cDNA |
| 36958_at | −6.2 | Cluster Incl. X95735: Homo sapiens |
| | | mRNA for zyxin |
| 37255_at | −6.2 | Cluster Incl. U36601: Homo sapiens |
| | | heparan N-deacetylase |
| 35239_at | −6.2 | Cluster Incl. X86810: Homo sapiens |
| | | EDMD gene |
| 37181_at | −60 | Cluster Incl. X76538: H. sapiens Mpv17 |
| | | mRNA |
| 36469_at | 10.8 | Cluster Incl. U46744: Human |
| | | dystrobrevin-alpha mRNA, complete cds |
| 33005_at | 10.4 | Cluster Incl. AF010144: Homo sapiens |
| | | neuronal thread protein AD7c-NTP |
| | | mRNA, complete cds |
| 41034_s_at | 8.8 | Cluster Incl. U92315: Homo sapiens |
| | | hydroxysteroid sulfotransferase |
| | | SULT2B1b (HSST2) mRNA |
| 33069_f_at | 5.4 | Cluster Incl. U06641: Human UDP |
| | | glucuronosyltransferase mRNA, partial |
| | | cds |
| 32641_at | 5.2 | Cluster Incl. AB023196: Homo sapiens |
| | | mRNA for KIAA0979 protein, partial cds |
| 31923_f_at | 5.2 | Cluster Incl. U60269: Human |
| | | endogenous retrovirus HERV-K(HML6) |
| | | proviral clone HML6.17 |
| | | putative polymerase and envelope |
| | | genes, partial cds, and 3LTR |
| 35545_at | 5.2 | Cluster Incl. AB018282: Homo sapiens |
| | | mRNA for KIAA0739 protein, partial cds |
| 41642_at | 5 | Cluster Incl. X75940: H. sapiens beta |
| | | glucuronidase pseudogene |
| 36494_at | 3.6 | Cluster Incl. AF058918: Homo sapiens |
| | | unknown mRNA |
| 1894_f_at | 3.6 | Neurofibromatosis 2 Tumor Suppressor |

PBMC + LPS + VVC vs PBMC + LPS + PBS

| | | | |
|---|---|---|---|
| 37635_at | −8 | Cluster Incl. L09190: Human trichohyalin | |
| | | (TRHY) gene, complete cds | |
| 41681_at | −3.8 | Cluster Incl. AB005289: Homo sapiens | |
| | | mRNA for ABC transporter 7 protein, | |
| | | complete cds | |
| 38712_at | −3.4 | Cluster Incl. AL035291: H. sapiens gene | |
| | | from PACs 125H23 and 105D12 | |
| 350_at | −3.2 | D28118 | DEFINITION = HUMDB1 |
| | | | Human mRNA for DB1, |
| | | | complete cds |
| 37361_at | −3.2 | Cluster Incl. AF010187: Homo sapiens | |
| | | FGF-1 intracellular binding protein | |
| | | (FIBP) mRNA, complete cds | |
| 33837_at | −3 | Cluster Incl. AF069765: Homo sapiens | |
| | | signal recognition particle 72 (SRP72) | |
| | | mRNA, complete cds | |
| 1452_at | −2.6 | DEFINITION = U24576 Homo sapiens | |
| | | breast tumor autoantigen (LMO4) | |
| | | mRNA, complete cds | |
| 32606_at | −2.2 | Cluster Incl. AA135683: zl10c08.r1 | |
| | | Homo sapiens cDNA, 5 end | |
| 37597_s_at | −2.2 | Cluster Incl. AF055006: Homo sapiens | |
| | | clone 24666 sec6 homolog mRNA, | |
| | | partial cds | |
| 39423_f_at | −2 | Cluster Incl. AJ000644: Homo sapiens | |
| | | mRNA for SPOP | |
| 1775_at | 5 | L24559 | DEFINITION = HUMDNSPOL |
| | | | A Homo sapiens DNA |
| | | | polymerase alpha mRNA, |
| | | | complete cds |
| 1341_at | 3.8 | X52056 | DEFINITION = HSSPI1 |
| | | | Human mRNA for spi-1 |
| | | | proto-oncogene |
| 1976_s_at | 3.4 | X06292 | DEFINITION = HSFESFPS |
| | | | Human c-fes |
| 1837_at | 3.2 | Ras-Like Protein Tc21 | |
| 37904_s_at | 3 | Cluster Incl. X66436: H. sapiens hsr1 | |
| | | mRNA (partial) | |
| 36709_at | 2.8 | Cluster Incl. Y00093: H. sapiens mRNA | |
| | | for leukocyte adhesion glycoprotein | |
| | | p150,95 | |
| 40552_s_at | 2.8 | Cluster Incl. AL049987: Homo sapiens | |
| | | mRNA; cDNA DKFZp564F112 (from | |
| | | clone DKFZp564F112) | |
| 33372_at | 2.6 | Cluster Incl. AI189226: qd04h11.x1 | |
| | | Homo sapiens cDNA, 3 end | |
| 35517_at | 2.6 | Cluster Incl. M12807: Human T-cell | |
| | | surface glycoprotein T4 mRNA, | |
| | | complete cds | |
| 1703_g_at | 2.6 | DEFINITION = S75174 E2F- | |
| | | 4 = transcription factor [human, Nalm6 | |
| | | and HeLa cells, mRNA, 1539 nt] | |

PBMC + PHA/IL2 vs PBMC + PBS

| | | | |
|---|---|---|---|
| 35961_at | −10.4 | Cluster Incl. AL049390: Homo sapiens | |
| | | mRNA; cDNA DKFZp586O1318 (from | |
| | | clone DKFZp586O1318) | |
| 33687_at | −9.8 | Cluster Incl. AL049782: Novel human | |
| | | gene mapping to chomosome 13 | |
| 40599_at | −8 | Cluster Incl. AL109669: Homo sapiens | |
| | | mRNA full length insert cDNA clone | |
| | | EUROIMAGE 31839 | |
| 41814_at | −8 | Cluster Incl. M29877: Human alpha-L- | |
| | | fucosidase, complete cds | |
| 33957_at | −7.8 | Cluster Incl. X81001: H. sapiens HCG II | |
| | | mRNA | |
| 39936_at | −7.8 | Cluster Incl. U95626: Homo sapiens | |
| | | ccr2b (ccr2), ccr2a (ccr2), ccr5 (ccr5) | |
| | | and ccr6 (ccr6) genes, | |
| | | complete cds, and lactoferrin | |
| | | (lactoferrin) gene, partial cds | |
| 41610_at | −7.4 | Cluster Incl. AB011105: Homo sapiens | |
| | | mRNA for KIAA0533 protein, partial cds | |
| 39938_g_at | −7 | Cluster Incl. U03905: Human monocyte | |
| | | chemoattractant protein 1 receptor | |
| | | (MCP-1RB) | |
| 37708_r_at | −6.6 | Cluster Incl. M81118: Human alcohol | |
| | | dehydrogenase chi polypeptide (ADH5) | |
| | | gene | |
| 37988_at | −5.4 | Cluster Incl. M89957: Human | |
| | | immunoglobulin superfamily member B | |
| | | cell receptor | |
| | | complex cell surface glycoprotein (IGB) | |
| 1021_at | 12.6 | J00219 | DEFINITION = HUMIFNG |
| | | | Human immune interferon |
| | | | (IFN-gamma) gene, complete |
| | | | cds |
| 431_at | 11 | X02530 | DEFINITION = HSINFGER |
| | | (with homology to platelet proteins) | Human mRNA for gamma- |
| | | | interferon inducible early |
| | | | response gene |
| 40702_at | 10.6 | Cluster Incl. X13274: Human mRNA for | |
| | | interferon IFN-gamma | |
| 37219_at | 10.6 | Cluster Incl. X72755: H. sapiens Humig | |
| | | mRNA | |
| 37279_at | 10.4 | Cluster Incl. U10550: Human Gem | |
| | | GTPase (gem) mRNA, complete cds | |
| 1579_at | 9.4 | M27288 | DEFINITION = HUMOCS3 |
| | | | Human oncostatin M gene, |
| | | | exon 3 |
| 1702_at | 9.4 | X01057 | DEFINITION = HSIL2REC |
| | | | Human mRNA for interleukin- |
| | | | 2 receptor |
| 1334_s_at | 9 | X03656 | DEFINITION = HSGCSFG |
| | | | Human gene for granulocyte |
| | | | colony-stimulating factor (G- |
| | | | CSF) |
| 38598_at | 8.2 | Cluster Incl. AI679353: tu73f03.x1 Homo | |
| | | sapiens cDNA, 3 end | |
| 1538_s_at | 8.2 | X00695 | DEFINITION = HSIL05 |
| | | | Human interleukin-2 (IL-2) |
| | | | gene and 5 -flanking region |

PBMC + PHA/IL2 + MTR vs PBMC + PHA/IL2 + PBS

| | | | |
|---|---|---|---|
| 38139_at | −7 | Cluster Incl. AF017445: Homo sapiens | |
| | | GDP-L-fucose pyrophosphorylase | |
| | | (GFPP) mRNA, complete cds | |
| 1602_at | −2 | L33881 | DEFINITION = HUMPKCI |
| | | | Human protein kinase C iota |
| | | | isoform, complete cds |
| 33936_at | −1.4 | Cluster Incl. D86181: Homo sapiens | |
| | | DNA for galactocerebrosidase | |
| 38035_at | −1.2 | Cluster Incl. AF072928: Homo sapiens | |
| | | myotubularin related protein 6 mRNA, | |
| | | partial cds | |
| 39685_at | −1.2 | Cluster Incl. AL050282: Homo sapiens | |
| | | mRNA; cDNA DKFZp586H2219 (from | |
| | | clone DKFZp586H2219) | |
| 40928_at | −1.2 | Cluster Incl. W26496: 30d2 Homo | |
| | | sapiens cDNA | |
| 1439_s_at | −1.2 | X75346 | DEFINITION = HSMAPKAP |
| | | | H. sapiens mRNA for MAP |
| | | | kinase activated protein |
| | | | kinase |
| 39969_at | −1.2 | Cluster Incl. AA255502: zr85b06.r1 | |
| | | Homo sapiens cDNA, 5 end | |
| 36100_at | −1 | Cluster Incl. AF022375: Homo sapiens | |
| | | vascular endothelial growth factor | |
| | | mRNA, complete cds | |
| 37975_at | −1 | Cluster Incl. X04011: Human mRNA of | |
| | | X-CGD gene involved in chronic | |
| | | granulomatous disease located | |
| | | on chromosome X | |
| 33715_r_at | 7.6 | Cluster Incl. U80017: Homo sapiens | |
| | | basic transcription factor 2 p44 (btf2p44) | |
| | | gene, partial cds, | |
| | | neuronal apoptosis inhibitory protein | |
| | | (naip) and survival motor neuron protein | |
| | | (smn) genes, complete cds | |
| 36230_at | 7.4 | Cluster Incl. AI624038: ts25h10.x1 Homo | |
| | | sapiens cDNA, 3 end | |
| 40945_at | 7.2 | Cluster Incl. AI991531: ws09g12.x1 | |
| | | Homo sapiens cDNA, 3 end | |
| 39260_at | 6.8 | Cluster Incl. U59185: Human putative | |
| | | monocarboxylate transporter (MCT) | |
| | | mRNA, complete cds | |
| 31810_g_at | 5.4 | Cluster Incl. Z21488: H. sapiens contactin | |
| | | mRNA | |
| 558_at | 5.2 | M98776 | DEFINITION = HUMKRT1X |
| | | | Human keratin 1 gene, |
| | | | complete cds |
| 1914_at | 4.6 | U66838 | DEFINITION = HSU66838 |
| | | | Human cyclin A1 mRNA, |
| | | | complete cds |
| 36423_at | 4.4 | Cluster Incl. W47047: zc38g10.r1 Homo | |
| | | sapiens cDNA, 5 end | |
| 1714_at | 4.2 | U26914 | DEFINITION = HSU26914 |
| | | | Human ras-responsive |
| | | | element binding protein |
| | | | (RREB-1) mRNA |
| 38942_r_at | 3.4 | Cluster Incl. W28610: 49b12 Homo | |
| | | sapiens cDNA | |

PBMC + PHA/IL2 + MTRV vs PBMC + PHA/IL2 + PBS

| | | | |
|---|---|---|---|
| 33148_at | −8.4 | Cluster Incl. AI459274: tk11f11.x1 Homo | |
| | | sapiens cDNA, 3 end | |
| 37626_at | −7.6 | Cluster Incl. D78261: Human ICSAT | |
| | | transcription factor mRNA, partial cds, | |
| | | similar to mouse Pip | |
| 1219_at | −7.4 | X15949 | DEFINITION = HSIRF2 |
| | | | Human mRNA for interferon |
| | | | regulatory factor-2 (IRF-2) |
| 37627_g_at | −6.6 | Cluster Incl. D78261: Human ICSAT | |
| | | transcription factor mRNA, partial cds, | |
| | | similar to mouse Pip | |
| 31491_s_at | −6.4 | Cluster Incl. X98175: H. sapiens mRNA | |
| | | for MACH-beta-2 protein | |
| 39142_at | −6 | Cluster Incl. AJ001810: Homo sapiens | |
| | | mRNA for pre-mRNA cleavage factor I | |
| | | subunit | |
| 1571_f_at | −5.6 | L49229 | DEFINITION = HUMRB1AAD |
| | | with a 3 bp deletion in exon 22 (L11910 | L Homo sapiens |
| | | bases 161855-162161) | retinoblastoma susceptibility |
| | | | protein (RB1) gene, |
| 37486_f_at | −5.2 | Cluster Incl. U68385: Human Meis1- | |
| | | related protein 2 (MRG2), mRNA, partial | |
| | | cds | |
| 32630_f_at | −5 | Cluster Incl. Y07827: H. sapiens mRNA | |
| | | for put. B7,3 molecule of CD80-CD60 | |
| | | protein family | |
| 32343_at | −4.4 | Cluster Incl. J03796: Human erythroid | |
| | | isoform protein 4.1 mRNA, complete cds | |
| 558_at | 7.2 | M98776 | DEFINITION = HUMKRT1X |
| | | | Human keratin 1 gene, |
| | | | complete cds |
| 40888_f_at | 3 | Cluster Incl. W28170: 43a12 Homo | |
| | | sapiens cDNA | |
| 40858_at | 5.4 | Cluster Incl. M34715: Human pregnancy- | |
| | | specific beta-1-glycoprotein mRNA | |
| | | PSG95, complete cds | |
| 40627_at | 8.2 | Cluster Incl. AI192108: qa06d10.x1 | |
| | | Homo sapiens cDNA, 3 end | |
| 40399_r_at | 4 | Cluster Incl. AI743406: wg92g12.x1 | |
| | | Homo sapiens cDNA, 3 end | |
| 39520_at | 3.4 | Cluster Incl. AI924382: wn60d01.x1 | |
| | | Homo sapiens cDNA, 3 end | |
| 37418_at | 5.8 | Cluster Incl. M36653: Human Oct-2 | |
| | | factor mRNA, complete cds | |
| 31961_r_at | 3.2 | Cluster Incl. AF070579: Homo sapiens | |
| | | clone 24487 mRNA sequence | |
| 31756_at | 5.4 | Cluster Incl. AL049328: Homo sapiens | |
| | | mRNA; cDNA DKFZp564E026 (from | |
| | | clone DKFZp564E026) | |
| 1894_f_at | 4 | Neurofibromatosis 2 Tumor Suppressor | |
| 38942_r_at | 3.4 | Cluster Incl. W28610: 49b12 Homo | |
| | | sapiens cDNA | |

PBMC + PHA/IL2 + MTRVLQGVLPALPQVVC vs PBMC + PHA/IL2 + PBS

| | | | |
|---|---|---|---|
| 1774_at | −7.6 | L06895 | DEFINITION = HUMMAD |
| | | | Homo sapiens antagonizer of |
| | | | myc transcriptional activity |
| | | | (Mad) Mrna |
| 1579_at | −6.6 | M27288 | DEFINITION = HUMOCS3 |
| | | | Human oncostatin M gene, |
| | | | exon 3 |
| 33524_at | −6.4 | Cluster Incl. X59656: H. sapiens crk-like | |
| | | gene CRKL | |
| 40968_at | −6.4 | Cluster Incl. AB004904: Homo sapiens | |
| | | mRNA for STAT induced STAT inhibitor- | |
| | | 3, complete cds | |
| 535_s_at | −6.2 | U20816 | DEFINITION = HSU20816 |
| | | | Human nuclear factor kappa- |
| | | | B2 (NF-KB2) gene, partial |
| | | | cds |
| 1226_at | −5.8 | U69611 | DEFINITION = HSU69611 |
| | | | Human TNF-alpha |
| | | | converting enzyme mRNA, |
| | | | complete cds |
| 37627_g_at | −5.8 | Cluster Incl. D78261: Human ICSAT | |
| | | transcription factor mRNA, partial cds, | |
| | | similar to mouse Pip | |
| 867_s_at | −5.6 | U12471 | DEFINITION = HSU12471 |
| | | | Human thrombospondin-1 |
| | | | gene, partial cds |
| 31858_at | −5.2 | Cluster Incl. X07315: Human gene for | |
| | | PP15 (placental protein 15) | |
| 40951_at | −5.2 | Cluster Incl. AL049250: Homo sapiens | |
| | | mRNA; cDNA DKFZp564D113 (from | |
| | | clone DKFZp564D113) | |
| 35950_at | 6.2 | Cluster Incl. U90841: Homo sapiens | |
| | | SSX4 (SSX4) mRNA, complete cds | |
| 41290_at | 6 | Cluster Incl. W27873: 39a11 Homo | |
| | | sapiens cDNA | |
| 37451_at | 5.6 | Cluster Incl. AL109695: Homo sapiens | |
| | | mRNA full length insert cDNA clone | |
| | | EUROIMAGE 39820 | |
| 31810_g_at | 4.2 | Cluster Incl. Z21488: H. sapiens contactin | |
| | | mRNA | |
| 31876_r_at | 4.2 | Cluster Incl. U92014: Human clone | |
| | | 121711 defective mariner transposon | |
| | | Hsmar2 mRNA sequence | |
| 37584_at | 3.8 | Cluster Incl. AJ007669: Homo sapiens | |
| | | mRNA for Fanconi anemia group G | |
| 618_at | 3.8 | M26167 | DEFINITION = HUMPF4V1A |
| | | | Human platelet factor 4 |
| | | | varation 1 (PF4var1) gene, |
| | | | complete cds |
| 32645_at | 3.8 | Cluster Incl. AB007946: Homo sapiens | |
| | | mRNA for KIAA0477 protein, complete | |
| | | cds | |
| 40888_f_at | 3.8 | Cluster Incl. W28170: 43a12 Homo | |
| | | sapiens cDNA | |
| 1894_f_at | 3.6 | Neurofibromatosis 2 Tumor Suppressor | |
| 1894_f_at | 4 | Neurofibromatosis 2 Tumor Suppressor | |
| 38942_r_at | 3.4 | Cluster Incl. W28610: 49b12 Homo | |
| | | sapiens cDNA | |

PBMC + MTRV vs PBMC + PBS

| | | |
|---|---|---|
| 34230_r_at | −4 | Cluster Incl. D84454: Human mRNA for |
| | | UDP-galactose translocator, complete |
| | | cds |
| 1927_s_at | −2.4 | Human activin receptor like kinase 1 |
| | | (ALK-1) gene; exon 10 and complete |
| | | cds |
| 34184_at | −2.4 | Cluster Incl. AB012162: Homo sapiens |
| | | mRNA for APCL protein, complete cds |
| 32856_at | −2.2 | Cluster Incl. AB020626: Homo sapiens |
| | | mRNA for KIAA0819 protein, partial cds |
| 31535_i_at | −2 | Cluster Incl. W27858: 39e3 Homo |
| | | sapiens cDNA |
| 40858_at | −2 | Cluster Incl. M34715: Human pregnancy- |
| | | specific beta-1-glycoprotein mRNA |
| | | PSG95, complete cds |
| 39298_at | −1.8 | Cluster Incl. AB022918: Homo sapiens |
| | | mRNA for alpha2,3-sialyltransferase |
| | | ST3Gal VI, complete cds |
| 33105_at | −1.8 | Cluster Incl. W28790: 54g3 Homo |
| | | sapiens cDNA |
| 36821_at | −1.8 | Cluster Incl. AL050367: Homo sapiens |
| | | mRNA; cDNA DKFZp564A026 (from |
| | | clone DKFZp564A026) |
| 39815_at | −1.8 | Cluster Incl. AA883101: am24d05.s1 |
| | | Homo sapiens cDNA, 3 end |
| 33876_at | 6.8 | Cluster Incl. AL050107: Homo sapiens |
| | | mRNA; cDNA DKFZp586I1419 (from |
| | | clone DKFZp586I1419) |
| 34610_at | 2.6 | Cluster Incl. W25845: 13h9 Homo |
| | | sapiens cDNA |
| 35934_at | 2.2 | Cluster Incl. L19161: Human translation |
| | | initiation factor eIF-2 gamma subunit |
| | | mRNA, complete cds |
| 39077_at | 2.2 | Cluster Incl. U41843: Human Dr1- |
| | | associated corepressor (DRAP1) |
| | | mRNA, complete cds |
| 41199_s_at | 1.6 | Cluster Incl. W27050: 19f7 Homo |
| | | sapiens cDNA |
| 38207_at | 1.4 | Cluster Incl. AW006742: wr28g10.x1 |
| | | Homo sapiens cDNA, 3 end |
| 38868_at | 1.4 | Cluster Incl. U43774: Human Fc alpha |
| | | receptor, splice variant FcalphaR a.2 |
| | | (CD89) mRNA, complete cds |
| 41291_at | 1.4 | Cluster Incl. AC004528: Homo sapiens |
| | | chromosome 19, cosmid R32184 |
| 33424_at | 1.2 | Cluster Incl. Y00281: Human mRNA for |
| | | ribophorin I |
| 35745_f_at | 1.2 | Cluster Incl. X78136: H. sapiens hnRNP- |
| | | E2 mRNA |

PBMC + AQGV vs PBMC + PBS

| | | |
|---|---|---|
| 33985_s_at | −8.2 | Cluster Incl. W28616: 49b9 Homo |
| | | sapiens cDNA |
| 40536_f_at | −8.2 | Cluster Incl. AI254524: qv48f07.x1 Homo |
| | | sapiens cDNA, 3 end |
| 32509_at | −6.6 | Cluster Incl. AI307607: tb15h10.x1 |
| | | Homo sapiens cDNA, 3 end |
| 40627_at | −5.8 | Cluster Incl. AI192108: qa06d10.x1 |
| | | Homo sapiens cDNA, 3 end |
| 32636_f_at | −5.8 | Cluster Incl. AB007881: Homo sapiens |
| | | KIAA0421 mRNA, partial cds |
| 31665_s_at | −5 | Cluster Incl. W27675: 36b3 Homo |
| | | sapiens cDNA |
| 40535_i_at | −4.2 | Cluster Incl. AI254524: qv48f07.x1 Homo |
| | | sapiens cDNA, 3 end |
| 1017_at | −4 | Human hMSH6 gene, 5 UTR and |
| 37758_s_at | −3.8 | Cluster Incl. W28479: 47d8 Homo |
| | | sapiens cDNA |
| 33986_r_at | −3.6 | Cluster Incl. W28616: 49b9 Homo |
| | | sapiens cDNA |
| 35578_at | 8.2 | Cluster Incl. AF070586: Homo sapiens |
| | | clone 24528 mRNA sequence |
| 33876_at | 6.6 | Cluster Incl. AL050107: Homo sapiens |
| | | mRNA; cDNA DKFZp586I1419 (from |
| | | clone DKFZp586I1419) |
| 38379_at | 2.4 | Cluster Incl. X76534: H. sapiens NMB |
| | | mRNA |
| 36927_at | 2.2 | Cluster Incl. AB000115: Homo sapiens |
| | | mRNA expressed in osteoblast, |
| | | complete cds |
| 34628_at | 2.2 | Cluster Incl. Y09321: H. sapiens |
| | | TAFII105 mRNA, partial |
| 300_f_at | 2 | Transcription Factor Btf3 Homolog |
| 31919_at | 2 | Cluster Incl. AF002986: Homo sapiens |
| | | platelet activating receptor homolog |
| | | (H963) mRNA, complete cds |
| 35372_r_at | 1.8 | Cluster Incl. M17017: Human beta- |
| | | thromboglobulin-like protein mRNA, |
| | | complete cds |
| 546_at | 1.8 | protein kinase inhibitor [human, |
| | | neuroblastoma cell line SH-SY-5Y, |
| | | mRNA, 2147 nt] |
| 41834_g_at | 1.6 | Cluster Incl. AB016492: Homo sapiens |
| | | hJTB gene, complete cds |

PBMC + LPS + AQGV vs PBMC + LPS + PBS

| | | |
|---|---|---|
| 1473_s_at | −8 | c-myb gene extracted from Human (c- |
| | | myb) gene, complete cds, and five |
| | | complete alternatively spliced |
| 38520_r_at | −7 | Cluster Incl. U35735: Human RACH1 |
| | | (RACH1) mRNA, complete cds |
| 32041_r_at | −2.8 | Cluster Incl. AB007892: Homo sapiens |
| | | KIAA0432 mRNA, complete cds |
| 32834_r_at | −2.6 | Cluster Incl. AF013591: Homo sapiens |
| | | homolog of the Aspergillus nidulans |
| | | sudD gene product Mrna |
| 32784_at | −2.2 | Cluster Incl. AB011108: Homo sapiens |
| | | mRNA for KIAA0536 protein, partial cds |
| 38902_r_at | −2.2 | Cluster Incl. X15875: Human mRNA for |
| | | cAMP response element (CRE-BP1) |
| | | binding protein |
| 36783_f_at | −2 | Cluster Incl. M55422: Human Krueppel- |
| | | related zinc finger protein (H-plk) |
| | | mRNA, complete cds |
| 32647_at | −1.8 | Cluster Incl. AF060902: Homo sapiens |
| | | vesicle soluble NSF attachment protein |
| | | receptor VTl2 mRNA |
| 37740_r_at | −1.8 | Cluster Incl. J02683: Human ADP |
| 31481_s_at | −1.8 | Cluster Incl. M92383: Homo sapiens |
| | | thymosin beta-10 gene, 3end |
| 36275_at | 7.8 | Cluster Incl. AB002438: Homo sapiens |
| | | mRNA from chromosome 5q21-22, |
| | | clone-FBR89 |
| 39635_at | 6.2 | Cluster Incl. AB023177: Homo sapiens |
| | | mRNA for KIAA0960 protein, partial cds |
| 41109_at | 5.8 | Cluster Incl. M31452: Human proline-rich |
| | | protein (PRP) mRNA, complete cds |
| 36754_at | 5.6 | Cluster Incl. X60435: H. sapiens gene |
| | | PACAP for pituitary adenylate cyclase |
| | | activating polypeptide |
| 37810_at | 5.2 | Cluster Incl. U82759: Human |
| | | homeodomain protein HoxA9 mRNA, |
| | | complete cds |
| 38856_at | 5 | Cluster Incl. AL109724: Homo sapiens |
| | | mRNA full length insert cDNA clone |
| | | EUROIMAGE 417629 |
| 39937_at | 4.8 | Cluster Incl. U03905: Human monocyte |
| | | chemoattractant protein 1 receptor |
| | | (MCP-1RB) |
| 40740_at | 3.8 | Cluster Incl. M93650: Human paired box |
| | | gene (PAX6) homologue, complete cds |
| 1177_at | 3.8 | Dna-Binding Protein Ap-2, Alt. Splice 3 |
| 36148_at | 3 | Cluster Incl. U48437: Human amyloid |
| | | precursor-like protein 1 mRNA, |
| | | complete cds |

PBMC + LPS + LQG vs PBMC + LPS + PBS

| | | |
|---|---|---|
| 39077_at | −1.8 | Cluster Incl. U41843: Human Dr1- |
| | | associated corepressor (DRAP1) |
| | | mRNA, complete cds |
| 41787_at | −1.8 | Cluster Incl. AI452442: tj62a07.x1 Homo |
| | | sapiens cDNA, 3 end |
| 40690_at | −1.6 | Cluster Incl. X54942: H. sapiens ckshs2 |
| | | mRNA for Cks1 protein homologue |
| 38567_at | −1.4 | Cluster Incl. L38820: Homo sapiens |
| | | HMC class I antigen-like glycoprotein |
| | | (CD1D) gene |
| 39519_at | −1.4 | Cluster Incl. AB014592: Homo sapiens |
| | | mRNA for KIAA0692 protein, partial cds |
| 32790_at | −1.4 | Cluster Incl. D59253: Human mRNA for |
| | | NCBP interacting protein 1, complete |
| | | cds |
| 39921_at | −1.4 | Cluster Incl. AI526089: DU3.2-7.H07.r |
| | | Homo sapiens cDNA, 5 end |
| 33389_at | −1.2 | Cluster Incl. U23942: Human lanosterol |
| | | 14-demethylase cytochrome P450 |
| | | (CYP51) mRNA, complete cds |
| 38676_at | −1.2 | Cluster Incl. AA059408: zl96e07.r1 |
| | | Homo sapiens cDNA, 5 end |
| 39969_at | −1.20 | Cluster Incl. AA255502: zr85b06.r1 |
| | | Homo sapiens cDNA, 5 end |
| 2034_s_at | 2.8 | Human cyclin-dependent kinase |
| | | inhibitor p27kip1 mRNA, complete cds. |
| 31346_at | 2.8 | Cluster Incl. AJ001481: Homo sapiens |
| | | mRNA for DUX1 protein |
| 41842_at | 2.4 | Cluster Incl. AI701156: we10f0g.x1 |
| | | Homo sapiens cDNA, 3 end |
| 33780_at | 2.2 | Cluster Incl. M36200: Human |
| | | synaptobrevin 1 (SYB1) gene |
| 34469_at | 2.2 | Cluster Incl. X84746: H. sapiens Histo- |
| | | blood group AB0 gene, exon 1 |
| 39459_at | 2.2 | Cluster Incl. W28765: 51d2 Homo |
| | | sapiens cDNA |
| 40013_at | 2.2 | Cluster Incl. Y12696: H. sapiens mRNA |
| | | homologous to the p64 bovine chloride |
| | | channel peptide |
| 39833_at | 2 | Cluster Incl. R54564: yg81b12.s1 Homo |
| | | sapiens cDNA, 3 end |
| 1562_g_at | 1.8 | Human protein-tyrosine phosphatase |
| | | mRNA, complete cds |
| 415_at | 1.8 | Homo sapiens crk-like gene CRKL |

PBMC + LPS + LQGV vs PBMC + LPS + PBS

| | | |
|---|---|---|
| 36342_r_at | −9.8 | Cluster Incl. X64877: H. sapiens mRNA |
| | | for serum protein |
| 36435_at | −4 | Cluster Incl. AF070670: Homo sapiens |
| | | protein phosphatase 2C alpha 2 mRNA, |
| | | complete cds |
| 32041_r_at | −3.8 | Cluster Incl. AB007892: Homo sapiens |
| | | KIAA0432 mRNA, complete cds |
| 39969_at | −3.6 | Cluster Incl. AA255502: zr85b06.r1 |
| | | Homo sapiens cDNA, 5 end |
| 39956_at | −3.2 | Cluster Incl. AF041853: Homo sapiens |
| | | kinesin family member protein KIF3A |
| | | mRNA, complete cds |
| 39226_at | −3 | Cluster Incl. X06026: H. sapiens CD3G |
| | | gene, exon 1 (and joined CDS) |
| 40604_at | −3 | Cluster Incl. Y13493: Homo sapiens |
| | | mRNA for protein kinase Dyrk2 |
| 32326_at | −2.8 | Cluster Incl. W27519: 31h8 Homo |
| | | sapiens cDNA |
| 37699_at | −2.6 | Cluster Incl. U29607: Human methionine |
| | | aminopeptidase mRNA, complete cds |
| 38520_r_at | −2.6 | Cluster Incl. U35735: Human RACH1 |
| | | (RACH1) mRNA, complete cds |
| 40934_at | 4.6 | Cluster Incl. W26097: 22f1 Homo |
| | | sapiens cDNA |
| 39930_at | 4.2 | Cluster Incl. D83492: Homo sapiens |
| | | mRNA for Eph-family protein, complete |
| | | cds |
| 31998_at | 3.8 | Cluster Incl. AJ012376: Homo sapiens |
| | | mRNA for ATP-binding cassette |
| | | transporter-1 (ABC-1) |
| 38660_at | 3.8 | Cluster Incl. F27891: HSPD16170 Homo |
| | | sapiens cDNA |
| 160028_s_at | 3.4 | X12949 Human ret proto-oncogene |
| | | mRNA for tyrosine kinase |
| 39547_at | 3 | Cluster Incl. AB008515: Homo sapiens |
| | | mRNA for RanBPM, complete cds |
| 38027_at | 3 | Cluster Incl. X53742: H. sapiens mRNA |
| | | for fibulin-1 B |
| 33085_at | 2.8 | Cluster Incl. U64863: Human hPD-1 |
| | | (hPD-1) mRNA, complete cds |
| 1976_s_at | 2.6 | HSFESFPS Human c-fes |
| 32608_at | 2.6 | Cluster Incl. AF000560: Homo sapiens |
| | | TTF-I interacting peptide 20 mRNA, |
| | | partial cds |

PBMC + LPS + MTR vs PBMC + LPS + PBS

| | | |
|---|---|---|
| 1227_g_at | −6 | Human TNF-alpha converting enzyme |
| | | mRNA; complete cds |
| 32834_r_at | −2.6 | Cluster Incl. AF013591: Homo sapiens |
| | | homolog of the Aspergillus nidulans |
| | | sudD gene product mRNA |
| 582_g_at | −2.6 | Human steroid receptor (TR2-11) |
| | | mRNA, complete cds |
| 31481_s_at | −2.2 | Cluster Incl. M92383: Homo sapiens |
| | | thymosin beta-10 gene, 3end |
| 35171_at | −2 | Cluster Incl. AB029006: Homo sapiens |
| | | mRNA for KIAA1083 protein, complete |
| | | cds |
| 41425_at | −2 | Cluster Incl. M98833: Human ERGB |
| | | transcription factor (FLI-1 homolog) |
| | | mRNA, complete cds |
| 39077_at | −1.8 | Cluster Incl. U41843: Human Dr1- |
| | | associated corepressor (DRAP1) |
| | | mRNA, complete cds |
| 40613_at | −1.8 | Cluster Incl. AL031775: dJ30M3.2 (novel |
| | | protein) |
| 34822_at | −1.6 | Cluster Incl. U58334: Human Bcl2, p53 |
| | | binding protein Bbp |
| 41495_at | −1.6 | Cluster Incl. W37606: zc12a03.r1 Homo |
| | | sapiens cDNA, 5 end |
| 38596_i_at | 8.6 | Cluster Incl. D50402: Human mRNA for |
| | | NRAMP1, complete cds |
| 39889_at | 7.8 | Cluster Incl. AI017532: ou35b04.x1 |
| | | Homo sapiens cDNA, 3 end |
| 1048_at | 7.8 | Human retinoid X receptor-gamma |
| | | mRNA, complete cds |
| 38027_at | 6 | Cluster Incl. X53742: H. sapiens mRNA |
| | | for fibulin-1 B |
| 32131_at | 5.6 | Cluster Incl. AB014575: Homo sapiens |
| | | mRNA for KIAA0675 protein, complete |
| | | cds |
| 35928_at | 5.2 | Cluster Incl. J02969: Human thyroid |
| | | peroxidase mRNA, clone phTPO-2.8 |
| 40238_at | 5 | Cluster Incl. AI674208: wc07f02.x1 |
| | | Homo sapiens cDNA, 3 end |
| 35794_at | 4 | Cluster Incl. AB023159: Homo sapiens |
| | | mRNA for KIAA0942 protein, partial cds |
| 39547_at | 3.6 | Cluster Incl. AB008515: Homo sapiens |
| | | mRNA for RanBPM, complete cds |
| 32879_at | 3.4 | Cluster Incl. AL080233: Homo sapiens |
| | | mRNA; cDNA DKFZp586L111 (from |
| | | clone DKFZp586L111) |

PBMC + LPS + MTRV vs PBMC + LPS + PBS

| | | |
|---|---|---|
| 36342_r_at | −6.6 | Cluster Incl. X64877: H. sapiens mRNA |
| | | for serum protein |
| 38520_r_at | −3.2 | Cluster Incl. U35735: Human RACH1 |
| | | (RACH1) mRNA, complete cds |
| 40951_at | −2.6 | Cluster Incl. AL049250: Homo sapiens |
| | | mRNA; cDNA DKFZp564D113 (from |
| | | clone DKFZp564D113) |
| 40877_s_at | −2.2 | Cluster Incl. AF041080: Homo sapiens |
| | | D15F37 pseudogene, S3 allele, mRNA |
| | | sequence |
| 36435_at | −2 | Cluster Incl. AF070670: Homo sapiens |
| | | protein phosphatase 2C alpha 2 mRNA, |
| | | complete cds |
| 34666_at | −1.8 | Cluster Incl. X07834: Human mRNA for |
| | | manganese superoxide dismutase (EC |
| | | 1.15.1.1) |
| 33859_at | −1.4 | Cluster Incl. U96915: Homo sapiens sin3 |
| | | associated polypeptide p18 (SAP18) |
| | | mRNA, complete cds |
| 1532_g_at | −1.4 | Human BRCA2 region; mRNA |
| | | sequence CG006 |
| 393_s_at | −1.4 | HSRNAML11 Homo sapiens mRNA for |
| | | an acute myeloid leukaemia protein |
| | | (3917 bp) |
| 40121_at | −1.2 | Cluster Incl. U58522: Human huntingtin |
| | | interacting protein (HIP2) mRNA, |
| | | complete cds |
| 33590_at | 6.8 | Cluster Incl. AJ011980: Homo sapiens |
| | | mRNA sequence, IMAGE clone 446411 |
| 38146_at | 5.8 | Cluster Incl. AB011107: Homo sapiens |
| | | mRNA for KIAA0535 protein, complete |
| | | cds |
| 37070_at | 5.2 | Cluster Incl. D14720: Homo sapiens |
| | | gene for peripheral myelin protein zero |
| | | (MZP) |
| 39547_at | 3.2 | Cluster Incl. AB008515: Homo sapiens |
| | | mRNA for RanBPM, complete cds |
| 31923_f_at | 2.8 | Cluster Incl. U60269: Human |
| | | endogenous retrovirus HERV-K(HML6) |
| | | proviral clone HML6.17 putative |
| | | polymerase and envelope genes, partial |
| | | cds, and 3LTR |
| 32131_at | 2.8 | Cluster Incl. AB014575: Homo sapiens |
| | | mRNA for KIAA0675 protein, complete |
| | | cds |
| 1505_at | 2.6 | Human thymidylate syntase (EC |
| | | 2.1.1.45) gene, complete cds |
| 33197_at | 2.4 | Cluster Incl. U39226: Human myosin |
| | | VIIA (USH1B) mRNA, complete cds |
| 1841_s_at | 2.4 | Proto-Oncogene N-Cym |
| 41639_at | 2.2 | Cluster Incl. D38553: Human mRNA for |
| | | KIAA0074 gene, partial cds |

PBMC + LPS vs PBMC + PBS

| | | |
|---|---|---|
| 37208_at | −12.8 | Cluster Incl. AJ001612: Homo sapiens |
| | | mRNA for L-3-phosphoserine- |
| | | phosphatase homologue |
| 41214_at | −12.4 | Cluster Incl. M58459: Human ribosomal |
| | | protein (RPS4Y) isoform mRNA, |
| | | complete cds |
| 38355_at | −10.6 | Cluster Incl. AF000984: Homo sapiens |
| | | dead box, Y isoform (DBY) mRNA, |
| | | alternative transcript 2 |
| 37583_at | −10.2 | Cluster Incl. U52191: Human SMCY (H-Y) |
| | | mRNA, complete cds |
| 35885_at | −9 | Cluster Incl. AF000986: Homo sapiens |
| | | Drosophila fat facets related Y protein |
| | | (DFFRY) mRNA, complete cds |
| 34477_at | −8.2 | Cluster Incl. AF000994: Homo sapiens |
| | | ubiquitous TPR motif, Y isoform (UTY) |
| | | mRNA, alternative transcript 3 |
| 38585_at | −7.6 | Cluster Incl. M91036: H. sapiens G- |
| | | gamma globin and A-gamma globin |
| | | genes, complete cdss |
| 1048_at | −7.6 | Human retinoid X receptor-gamma |
| | | mRNA, complete cds |
| 1927_s_at | −7.6 | Human activin receptor like kinase 1 |
| | | (ALK-1) gene; exon 10 and complete |
| | | cds |
| 39957_at | −7.2 | Cluster Incl. AF150247: AF150247 |
| | | Homo sapiens cDNA |
| 38299_at | 16.4 | Cluster Incl. X04430: Human IFN-beta |
| | | 2a mRNA for interferon-beta-2 |
| 36543_at | 15.8 | Cluster Incl. J02931: Human placental |
| | | tissue factor (two forms) mRNA, |
| | | complete cds |
| 40385_at | 11.6 | Cluster Incl. U64197: Homo sapiens |
| | | chemokine exodus-1 mRNA, complete |
| | | cds |
| 34022_at | 11.6 | Cluster Incl. M36821: Human cytokine |
| | | (GRO-gamma) mRNA, complete cds |
| 1069_at | 11.6 | Human cyclooxygenase-2 (hCox-2) |
| | | gene, complete cds |
| 37187_at | 11 | Cluster Incl. M36820: Human cytokine |
| | | (GRO-beta) mRNA, complete cds |
| 38446_at | 9.4 | Cluster Incl. X56199: Human XIST, |
| | | coding sequence a mRNA (locus |
| | | DXS399E) |
| 36103_at | 9.4 | Cluster Incl. D90144: Homo sapiens |
| | | gene for LD78 alpha precursor, |
| | | complete cds |
| 39402_at | 8.8 | Cluster Incl. M15330: Human interleukin |
| | | 1-beta (IL1B) mRNA, complete cds |
| 1520_s_at | 8.4 | HUMEDN1B Homo sapiens endothelin- |
| | | 1 (EDN1) gene; complete cds. |

PBMC + LQG vs PBMC + PBS

| | | |
|---|---|---|
| 37469_at | −7.6 | Cluster Incl. D79988: Human mRNA for |
| | | KIAA0166 gene, complete cds |
| 31683_at | −6.6 | Cluster Incl. S71020: THRA1 |
| 1207_at | −6.2 | Homo sapiens mRNA PLSTIRE for |
| | | serine |
| 33359_at | −4.8 | Cluster Incl. AB018311: Homo sapiens |
| | | mRNA for KIAA0768 protein, partial cds |
| 39460_g_at | −3.4 | Cluster Incl. W28765: 51d2 Homo |
| | | sapiens cDNA |
| 1177_at | −3.4 | Dna-Binding Protein Ap-2, Alt. Splice 3 |
| 39831_at | −3.2 | Cluster Incl. AI972631: wr41c07.x1 |
| | | Homo sapiens cDNA, 3 end |
| 40501_s_at | −3 | Cluster Incl. X73114: H. sapiens mRNA |
| | | for slow MyBP-C |
| 34112_r_at | −2.6 | Cluster Incl. AL050065: Homo sapiens |
| | | mRNA; cDNA DKFZp566M043 (from |
| | | clone DKFZp566M043) |
| 41072_at | −2.4 | Cluster Incl. AF043101: Homo sapiens |
| | | caveolin-3 mRNA, complete cds |
| 35875_at | 3.6 | Cluster Incl. AJ011304: Homo sapiens |
| | | mRNA for sphingosine-1-phosphate |
| | | lyase, partial |
| 33435_r_at | 2.4 | Cluster Incl. AI525962: DU145-2.B11.r |
| | | Homo sapiens cDNA, 5 end |
| 33009_at | 2.2 | Cluster Incl. AF042838: Homo sapiens |
| | | MEK kinase 1 (MEKK1) mRNA, partial |
| | | cds |
| 32815_at | 2 | Cluster Incl. AI687419: tp95h03.x1 |
| | | Homo sapiens cDNA, 3 end |
| 1397_at | 2 | Human protein kinase (MLK-3) mRNA, |
| | | complete cds |
| 38684_at | 1.8 | Cluster Incl. AJ010953: Homo sapiens |
| | | mRNA for putative Ca2+-transporting |
| | | ATPase, partial |
| 37006_at | 1.8 | Cluster Incl. AI660656: wf23c07.x1 |
| | | Homo sapiens cDNA, 3 end |
| 1272_at | 1.6 | Human translation initiation factor eIF-2 |
| | | gamma subunit mRNA, complete cds |
| 40476_s_at | 1.6 | Cluster Incl. U58198: Human interleukin |
| | | enhancer binding factor 3 mRNA, |
| | | complete cds |
| 35167_at | 1.6 | Cluster Incl. AB007893: Homo sapiens |
| | | KIAA0433 mRNA, partial cds |

PBMC + LQGV vs PBMC + PBS

| | | |
|---|---|---|
| 32141_at | −7.8 | Cluster Incl. AB028995: Homo sapiens |
| | | mRNA for KIAA1072 protein, complete |
| | | cds |
| 1207_at | −6.2 | Homo sapiens mRNA PLSTIRE for |
| | | serine |
| 34517_at | −5.8 | Cluster Incl. X66435: H. sapiens mRNA |
| | | for HMG-CoA-synthase |
| 37567_at | −4 | Cluster Incl. X98834: H. sapiens mRNA |
| | | for zinc finger protein, Hsal2 |
| 1048_at | −2.2 | Human retinoid X receptor-gamma |
| | | mRNA, complete cds |
| 33699_at | −2 | Cluster Incl. M18667: Human |
| | | pepsinogen C gene |
| 1927_s_at | −1.8 | Human activin receptor like kinase 1 |
| | | (ALK-1) gene; exon 10 and complete |
| | | cds |
| 38254_at | −1.8 | Cluster Incl. AB020689: Homo sapiens |
| | | mRNA for KIAA0882 protein, partial cds |
| 34112_r_at | −1.8 | Cluster Incl. AL050065: Homo sapiens |
| | | mRNA; cDNA DKFZp566M043 (from |
| | | clone DKFZp566M043) |
| 34936_at | −1.6 | Cluster Incl. AB012130: Homo sapiens |
| | | SBC2 mRNA for sodium bicarbonate |
| | | cotransporter2, complete cds |
| 33628_g_at | 3.8 | Cluster Incl. U57843: Human |
| | | phosphatidylinositol 3-kinase delta |
| | | catalytic subunit mRNA, complete cds |
| 39077_at | 2.8 | Cluster Incl. U41843: Human Dr1- |
| | | associated corepressor (DRAP1) |
| | | mRNA, complete cds |
| 1272_at | 2.4 | Human translation initiation factor elF-2 |
| | | gamma subunit mRNA, complete cds |
| 1694_s_at | 2.2 | HUMTA120 Human mRNA for tumor- |
| | | associated 120 kDa nuclear protein |
| | | p120; partial cds(carboxyl terminus) |
| 41199_s_at | 1.8 | Cluster Incl. W27050: 19f7 Homo |
| | | sapiens cDNA |
| 35934_at | 1.8 | Cluster Incl. L19161: Human translation |
| | | initiation factor elF-2 gamma subunit |
| | | mRNA, complete cds |
| 41291_at | 1.8 | Cluster Incl. AC004528: Homo sapiens |
| | | chromosome 19, cosmid R32184 |
| 1226_at | 1.6 | Human TNF-alpha converting enzyme |
| | | mRNA; complete cds |
| 38868_at | 1.6 | Cluster Incl. U43774: Human Fc alpha |
| | | receptor, splice variant FcalphaR a.2 |
| | | (CD89) mRNA, complete cds |
| 1994_at | 1.4 | Human CRE-BP1 transcription factor |
| | | mRNA; complete cds |

PBMC + MTR vs PBMC + PBS

| | | |
|---|---|---|
| 35462_at | −7.2 | Cluster Incl. U17033: Human 180 kDa |
| | | transmembrane PLA2 receptor mRNA, |
| | | complete cds |
| 31683_at | −4 | Cluster Incl. S71020: THRA1 |
| 39315_at | −3.6 | Cluster Incl. D13628: Human mRNA for |
| | | KIAA0003 gene, complete cds |
| 34416_at | −2.6 | Cluster Incl. X57110: Human mRNA for |
| | | c-cbl proto-oncogene |
| 32565_at | −2.2 | Cluster Incl. U66619: Human SWI |
| 33105_at | −2 | Cluster Incl. W28790: 54g3 Homo |
| | | sapiens cDNA |
| 32336_at | −1.8 | Cluster Incl. X05236: Human fibroblast |
| | | mRNA for aldolase A |
| 271_s_at | −1.8 | Human cathepsin E mRNA, complete |
| | | cds |
| 38686_at | −1.6 | Cluster Incl. X71490: H. sapiens mRNA |
| | | for vacuolar proton ATPase, subunit D |
| 31810_g_at | −1.60 | Cluster Incl. Z21488: H. sapiens contactin |
| | | mRNA |
| 32926_at | 6.2 | Cluster Incl. AL049991: Homo sapiens |
| | | mRNA; cDNA DKFZp564G222 (from |
| | | clone DKFZp564G222) |
| 37464_at | 5 | Cluster Incl. AF048755: Homo sapiens |
| | | HsPex13p (PEX13) mRNA, complete |
| | | cds |
| 34233_i_at | 4.4 | Cluster Incl. AI688640: wd40b07.x1 |
| | | Homo sapiens cDNA, 3 end |
| 41121_at | 4 | Cluster Incl. AA203345: zx56b04.r1 |
| | | Homo sapiens cDNA, 5 end |
| 36732_at | 3.2 | Cluster Incl. AI004207: ot94g05.x1 |
| | | Homo sapiens cDNA, 3 end |
| 1272_at | 2.8 | Human translation initiation factor elF-2 |
| | | gamma subunit mRNA, complete cds |
| 35934_at | 2.6 | Cluster Incl. L19161: Human translation |
| | | initiation factor elF-2 gamma subunit |
| | | mRNA, complete cds |
| 39302_at | 2.6 | Cluster Incl. X56807: Human DSC2 |
| | | mRNA for desmocollins type 2a and 2b |
| 1694_s_at | 2.4 | HUMTA120 Human mRNA for tumor- |
| | | associated 120 kDa nuclear protein |
| | | p120; partial cds(carboxyl terminus) |
| 38207_at | 2.4 | Cluster Incl. AW006742: wr28g10.x1 |
| | | Homo sapiens cDNA, 3 end |

### Example IX

Analysis of different peptides, which may optionally be in a database or use an exisiting database, may be conducted using: proteomics tools and/or sequence alignment tools, such as BLAST database (ExPasy, NCBI), SMART (EMBL), and PATTINPROT (PBIL); Post-translational modification prediction tools, for example, SignalP (CBS), Primary structure analysis; HLA Peptide Binding Predictions (e.g., BIMAS); Prediction of MHC type I and II peptide binding (e.g., SYFPEITHI); Amino acid scale representation (which may measure hydrophobicity, and other conformational parameters) (e.g., PROTSCALE); and Representations of a protein fragment as a helical wheel (e.g., HelixWheel/HelixDraw).

All references, including database accession numbers, publications, patents, and patent applications, cited herein are hereby incorporated by reference to the same extent as if each reference were individually and specifically indicated to be incorporated by reference and were set forth in its entirety herein.

1. A method of creating a peptide database, said method comprising:
providing a first source of peptides;
screening a peptide to determine the activity of the peptide;
analyzing the results;
characterizing a physical, chemical, or biological property of the peptides;
recording in a peptide database the characteristics of all peptides;
recording in the peptide database results of said analyzing the peptides, thereby generating a database or part of a database of peptides.

2. The method according to claim 1, wherein said peptides are related to gonadotropin.

3. The method according to claim 1, wherein said peptides are between three and four amino acids in length.

4. The method according to claim 1, wherein said step of characterizing a physical, chemical, or biological property of the peptides comprises determining an effect of the peptide on nitrous oxide production in a cell.

5. The method according to claim 1, wherein said step of characterizing a physical, chemical, or biological property of the peptides comprises conducting a glucose tolerance test.

6. The method for creating a peptide database according to claim 1, further comprising aligning a sequence of a subset of the peptides.

7. The method for creating a peptide database according to claim 6, further comprising determining a consensus sequence.

8. The method for creating a peptide database according to claim 6, further comprising determining positional information.

9. The method according to claim 6, wherein the peptides are from two different species.

10. The method according to claim 1, wherein said peptides are related to human chorionic gonadotropin.

11. The method according to claim 1, wherein analyzing the peptides comprises analyzing an anti-shock effect.

12. The method according to claim 1, wherein analyzing the peptides comprises analyzing TNF-α.

13. The method according to claim 1, wherein analyzing the peptides comprises analyzing an affect of a peptide on angiogenesis.

14. The method according to claim 1, wherein analyzing the peptides comprises analyzing a condition associated with a dysfunctional LDL receptor.

15. The method according to claim 1, wherein analyzing the peptides comprises analyzing the affect of the peptide on at least one inflammatory mediator selected from the group consisting of IL-1-α, IL-1-β, IL-6, TNF-α, LIF, IFN-γ, OSM, CNTF, GM-CSF, IL-11, IL-12, IL-17, IL-18 and IL-8.

16. The method according to claim 1, wherein said peptide database further comprises annotational information relating to said polynucleotide sequences

17. The method according to claim 16, wherein said annotational information comprises at least one of origin, source, features and references for said peptides.

18. The method according to claim 1, further comprising searching the peptide database for a peptide having specified characteristics.

19. The method according to claim 18, wherein searching the peptide database is performed from a remote location.

20. The method according to claim 1, further comprising identifying one or more proteins having immunoregulatory activity and/or gene regulatory activity.

21. The method according to claim 1, wherein screening a peptide to determine the activity of the peptide comprises screening a peptide array.

22. The method according to claim 1, wherein said peptides are related to Beta-catenin.

23. The method according to claim 1, wherein said peptides are related to C-reactive protein.

24. The method according to claim 1, wherein said peptides are related to matrix metalloproteinase-2.

25. The method according to claim 1, wherein said peptides are related to Bruton's tyrosine kinase.

26. A method implemented in a computer system for presenting biomolecular sequence data, said method comprising:
retrieving protein characteristic data from a database in response to a user query; and
graphically depicting elements of the protein characteristic data in a user interface for said computer system, wherein said graphical depiction comprises at least one panel graphically depicting peptide sequence or composition information.

27. A method of producing a pharmaceutical, said method comprising:
determining the identity of a compound that modulates an activity selected from the group consisting of development of the systemic inflammatory response, release of other inflammatory mediators, regulation of members of the nuclear factor-κB family, accentuation or protection from sepsis, nitrate production, nitric oxide production, glucose tolerance and combinations thereof;
conducting therapeutic profiling of the compound for efficacy and toxicity in animals; and
formulating a pharmaceutical preparation including one or more compounds identified as having an acceptable therapeutic profile.

28. The method according to claim 27, further comprising establishing a distribution system for distributing the pharmaceutical preparation.

29. The method according to claim 28, further comprising establishing a sales group for marketing the pharmaceutical preparation.

30. The method according to claim 27, wherein determining the identity of a compound comprises searching a peptide database.

31. An improvement in a method of screening a candidate compound for biological activity by screening the compound in a cell line, the improvement comprising:
screening said candidate compound in a cell line wherein said cell line has been contacted with at least one exogenously added biologically active peptide having gene regulatory activity.

32. The method according to claim 31, wherein the at least one exogenously added biologically active peptide is selected from the list of peptides present in Table 1, Table 2, Table 3, Table 4, Table 5, or Table 6.

33. A method of identifying biologically active peptide fragments comprising:
identifying a protein that is subsequently cleaved in a subject's body by a peptidase to form a peptide having an activity as well as peptide fragments, and analyzing said peptide fragments for biological activity.

34. The method according to claim 33 wherein the biological activity is gene regulatory activity.

35. The method according to claim 33, wherein the protein is hCG.

36. The method according to claim 33, wherein the biological activity of the peptide fragments is different than the activity of the activity of the peptide.

37. The method according to claim 33, wherein the protein is Beta-catenin.

38. The method according to claim 33, wherein the protein is C-reactive protein.

39. The method according to claim 33, wherein the protein is matrix metalloproteinase-2.

40. The method according to claim 33, wherein the protein is Bruton's tyrosine kinase.

41. The method according to claim 33, wherein the peptide fragments are timers or tetramers.

42. The method according to claim 41, further comprising entering data obtained from analyzing said peptide fragments for biological activity into a database.

43. The method according to claim 33 wherein biological activity is determined by contacting a cell or cell culture with a peptide fragment and determining the gene expression profile of said cell or cell culture.

44. The method according to claim 43, wherein said expression profile is compared with the expression profile of a control cell or cell culture not having been contacted with said peptide fragment.

45. The method according to claim 43, further comprising generating a database describing genes up-regulated or down-regulated by said peptide fragment.

46. A database generated by a method according to claim 45.
